(19) United States
(12) Patent Application Publication
(10) Pub. No.: US 2017/0145058 A1
(43) Pub. Date: May 25, 2017

(54) METHOD FOR TARGETED CONJUGATION OF PEPTIDES AND PROTEINS BY PAIRED C2 BRIDGING OF CYSTEINE AMINO ACIDS

(71) Applicant: BAYER PHARMA AKTIENGESELLSCHAFT, Berlin (DE)

(72) Inventors: Nils GRIEBENOW, Dormagen (DE); Stefan BRÄSE, Troisdorf (DE); Alicia DILMAC, Wuppertal (DE)

(21) Appl. No.: 15/309,991

(22) PCT Filed: May 5, 2015
(86) PCT No.: PCT/EP2015/059804

§ 371 Date: Nov. 9, 2016

(30) Foreign Application Priority Data
May 9, 2014 (EP) 14167772.4

(51) Int. Cl. C07K 7/64; C07K 16/00

(52) U.S. Cl. CPC C07K 7/64; A61K 47/48369; C07K 16/00; A61K 38/00

(57) ABSTRACT

The present application relates to a novel process for the targeted conjugation of peptides and proteins which is characterized by the pairwise C2-bridging of cysteine amino acids via their thiol groups, furthermore to the conjugates of peptides and proteins which can be obtained by such a process and also to the use of such conjugates for the diagnosis and/or treatment of disorders.

The present application relates to a novel process for the targeted conjugation of peptides and proteins which is characterized by the pairwise C2-bridging of cysteine amino acids via their thiol groups, furthermore to the conjugates of peptides and proteins which can be obtained by such a process and also to the use of such conjugates for the diagnosis and/or treatment of disorders.

In recent years, peptide and protein conjugation has achieved enormous significance in the provision of therapeutically important active compounds, in the diagnosis of disease processes and in the elucidation of biochemical mechanisms [Flygare et al., 2013; Jones et al., 2013].

Essentially, by conjugation the properties of peptides and proteins are changed or modulated. Therapeutically relevant peptides and proteins can be conjugated, for example, with biocompatible polymers to prolong the half-life of the peptide or protein in question in plasma circulation and thus its duration of action, or to counteract possible immunogenicity [Veronese and Maro, 2008; Roberts et al., 2002]. Furthermore, peptides and proteins can be conjugated with biochemical markers, dyes or reactive groups which then, after administration, provide an insight into binding processes in certain organs or cell regions [Miller and Cornish, 2005].

A further field which attracts wide research interest are peptide and protein conjugates with active compounds which direct these active compounds in a targeted manner in certain cell or organ regions to the intended site of action (drug targeting). A prominent example are antibody drug conjugates (ADCs) which bind in a targeted manner to certain domains/antigens and, after internalisation into the cell and subsequent processing (for example in the lysosomes), release the active compound in the compartment of the site of action [Garnett, 2001; Wu and Senter, 2005; Sapra et al., 2011; Chari et al., 2014; Klinguer-Hamour et al., 2014; Tian et al., 2014].

There are numerous methods for conjugation of peptides or proteins. Thus, peptides and proteins can be provided by various processes with reactive groups which then for their part serve as point of attachment for active compounds or diagnostics [Ramil and Lin, 2013]. In addition, peptides and proteins can be conjugated via the functionalities of their amino acids [Widdison et al., 2006]. Here, the challenge is the provision of a method which allows a selective reaction of the desired functionality in the peptide or protein in the presence of the other generally unprotected (free) amino acid groups.

A further possibility consists in the generation by biochemical transformations of reactive groups which are accessible to subsequent conjugation. A prominent example is the reduction of one or more disulphide bonds formed by cysteines in a peptide or protein, in order to subsequently conjugate the thiol groups thus released. This may be affected by conjugation of a single thiol, preferably with maleinimides [Ghosh et al., 1990], or by bridging the two thiol groups of the former disulphide bond. Double Michael acceptors, for example, have been described for such a bridging of thiols, which lead to C1 or C3 bridging [Liberatore et al., 1990; Godwin et al., Int. Pat. Appl. WO 2005/007197-A2, WO 2010/100430-A1], and also bifunctional electrophilic maleinimides, which allow C2 bridging [Schumacher et al., 2013]:

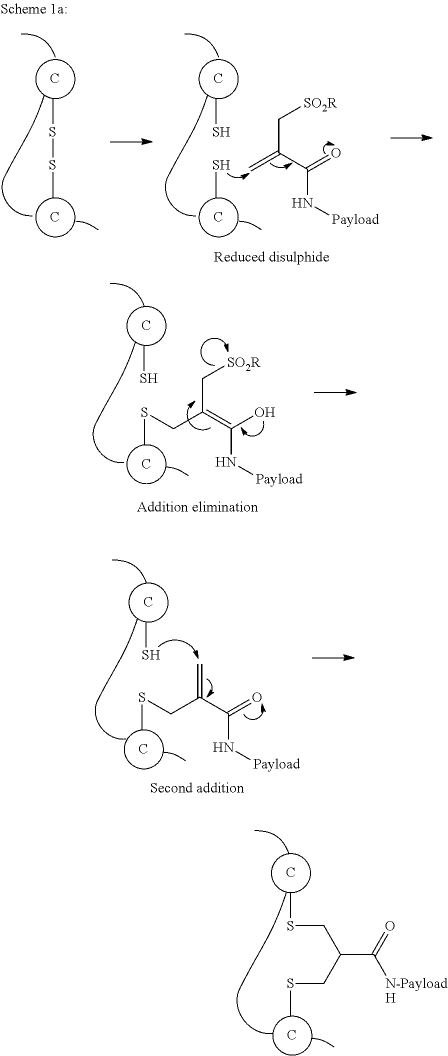

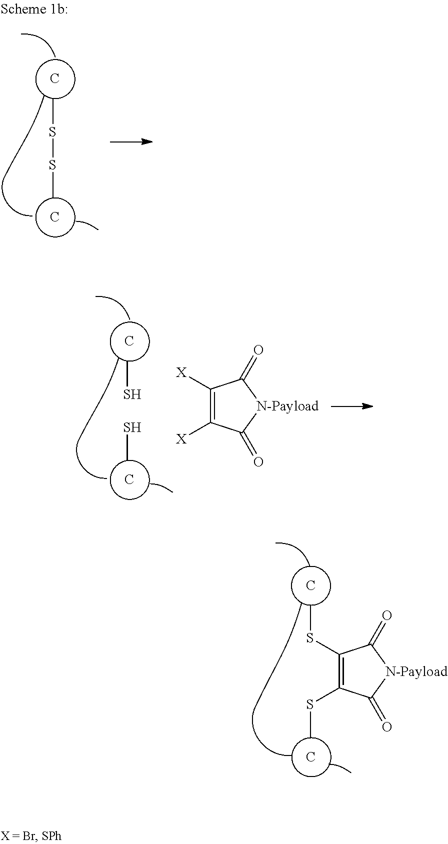

Scheme 1:

Bis-reactive conjugation reagents: a) “equilibrium transfer alkylating cross-link” (ETAC) reagent for disulphide C3 bridging; b) functional maleinimides for disulphide C2 bridging.

The methods outlined in Schemes 1a and 1b have been successfully used for providing antibody drug conjugates (ADCs). Here, interchain disulphide bridges of the antibody in question are reduced to free thiol groups and then conjugated in a bridging manner.

In the context of the present invention, we have now found a method which opens up a novel access to C2-bridged peptide and protein conjugates. In this method, the two thiols are, after reduction of the disulphide bridge formed via cysteines, reacted selectively with alkynes in a so-called thiol-yne reaction:

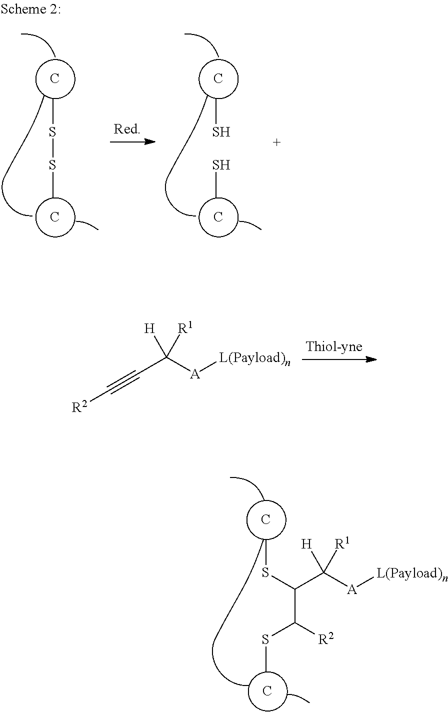

Scheme 2:

Thiol-yne reaction for C2-bridging of reduced disulphide bonds in peptides and proteins.

Thiol-yne reactions [thiol-yne coupling (TYC)] on peptides and proteins are known per se. However, hitherto they have not been applied to the C2-bridging conjugation to cysteines having free thiol groups, rather, each thiol group was conjugated separately, and not in a bridging manner [Lo Conte et al., 2011; Lo Conte et al., 2010; Massi and Nanni, 2012; Minozzi et al., 2011; Krannig et al., 2013; Aimetti et al., 2010]. Also known are thiol-yne reactions on peptides, which reactions serve to synthesize cyclopeptides by substituting a linear precursor peptide at a suitable position with an alkyne group which then reacts with a free cysteine of the same peptide with cyclization [Aimetti et al., Int. Pat. Appl. WO 2011/156686-A2]. The vinyl sulphide formed in this reaction may, in a subsequent reaction step, be reacted with a further (different) thiol. However, in contrast to the method according to the invention described herein, it is not the case that two free cysteines of a peptide or protein are bridged with one another, but in each case a peptide- or protein-bound alkyne is reacted with a cysteine of this peptide or protein. Also described in the literature is a bridging thiol-yne reaction of two connected mercaptoacetic esters having free thiol groups for the synthesis of macrocyclic crown ether and rotaxane structures [Zhou et al., 2010]. Described are furthermore thiol-yne reactions with peptides and proteins which serve to construct three-dimensional networks such as hydrogels [Anseth et al., Int. Pat. Appl. WO 2012/103445-A2; Kazantsev et al., US Pat. Appl. US 2014/0273153-A1]. In contrast to the method according to the invention described here, this does not yield homogeneous conjugates, but heterogeneous polymeric networks.

An essential feature of the cysteine bridging described above is that the stability or conformation of the peptide or protein which was provided beforehand by the corresponding disulphide bridge is substantially retained. Furthermore, in many cases a C2 bridge preserves the functionality, i.e. the affinity to the biological target of the peptide or protein [Jones et al., 2012; Gerona-Navarro et al., 2011].

On the other hand, Michael adducts such as in the case of the C1- or C3-bridged conjugates described above (see Scheme 1a) are known to be able to undergo, under physiological conditions, retro and exchange reactions with other thiol compounds such as, for example, glutathione or serum albumin, and thus be subject to a certain degradation in plasma circulation [Baldwin and Kiick, 2011; Toda et al., 2013; Shen et al., 2012]. To suppress this frequently undesired property, subsequent chemical transformations are required. In the case of the conjugates C2-bridged via maleinimide (see Scheme 1b), opening of the maleinimide or in the saturated case of the succinimide is required to achieve conjugation stable to thiols [Castaneda et al., 2013]. The compounds which can be obtained by the process according to the invention via thiol-yne reaction are not conjugates of Michael acceptors; accordingly, such retro and exchange reactions are not to be expected in this case.

Against the background shown, it was an object of the present invention to provide a novel method by which peptides and proteins can be conjugated selectively via their cysteine groups and in a stable form, thus being of use for the preparation of various defined peptide and protein conjugates.

To achieve this object, the invention provides a process which allows a conjugating C2-bridging of cysteines having free thiol groups in peptides and proteins via a selective thiol-yne reaction with alkyne derivatives. Appropriate free thiols can be generated, for example, by reducing disulphide bonds. Furthermore, the alkyne derivatives can be provided in a suitable manner with substituents, functional groups and/or linker units, allowing a corresponding modulation of the molecular properties of the target conjugates.

The present invention provides a process for preparing homogeneous peptide and protein conjugates, which process is characterized in that a peptide or protein of the formula (II)

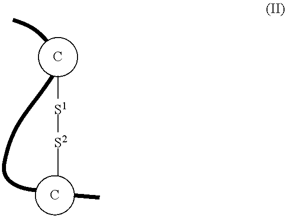

in which S¹ and S² represent cysteine sulphur atoms of this peptide or protein which are bonded in a disulphide bridge is converted under reducing conditions into a peptide or protein of the formula (III)

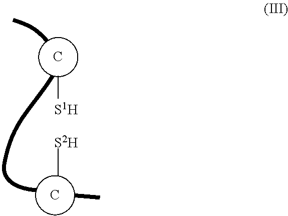

and this is then reacted under free-radical reaction conditions with an alkyne derivative of the formula (IV)

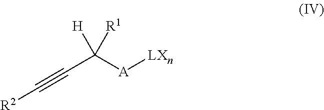

in which
R¹ and R² independently of one another represent hydrogen, alkyl, cycloalkyl, heterocycloalkyl, aryl, heteroaryl, hydroxy, alkoxy, amino, alkylamino, dialkylamino, hydroxycarbonyl, alkoxycarbonyl, alkylcarbonylamino or alkoxycarbonylamino,
   where alkyl, cycloalkyl, heterocycloalkyl, aryl, heteroaryl, alkoxy, alkylamino, dialkylamino, alkoxycarbonyl, alkylcarbonylamino and alkoxycarbonylamino for their part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino,
A represents a bond or a hydrocarbon chain having 1 to 100 carbon atoms from alkylene, cycloalkylene and/or arylene groups which may be interrupted once or more than once by identical or different groups selected from the group consisting of —O—, —S—, —S(═O)—, —S(═O)₂—, —NH—, —N(CH₃)—, —C(═O)—, —NH—C(═O)—, —C(═O)—NH—, —O—C(═O)—, —C(═O)—O—, —SO₂—NH—, —NH—SO₂—, —NH—NH—, —SO₂—NH—NH—, —NH—NH—SO₂—, —C(═O)—NH—NH—, —NH—NH—C(═O)—, —NH—C(═O)—NH—, —O—C(═O)—NH—, —NH—C(═O)—O— and a 4- to 10-membered aromatic or non-aromatic heterocycle having up to 4 heteroatoms from the group consisting of N, O, S, S(═O) and S(═O)₂,
or
R² and A are attached to one another and together with the interjacent carbon atoms form an 8-membered carbocycle which may be fused to a 3- to 6-membered cycloalkyl ring,
   where the 8-membered carbocycle and optionally the fused cycloalkyl ring may be mono- or polysubstituted by identical or different substituents from the group consisting of fluorine, alkyl, hydroxy, hydroxyalkyl and alkoxy,
L represents a bond or a linker,
X may be present n-times and represents an active compound molecule, a polymer, an alkaloid, a peptide, a protein, a carbohydrate, a nucleotide, a nucleoside, a steroid, a terpene, a porphyrin, a chlorin, a corrin, an eicosanoid, a pheromone, a vitamin, a biotin, a dye molecule or a cryptand or represents hydrogen, hydroxy, alkoxy, amino, alkylamino, dialkylamino, hydroxycarbonyl, alkoxycarbonyl, alkylcarbonylamino, alkoxycarbonylamino, alkyl, cycloalkyl, heterocycloalkyl, aryl or heteroaryl,
   where alkyl for its part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino
   and
   where cycloalkyl, heterocycloalkyl, aryl and heteroaryl for their part may be mono- or polysubstituted by identical or different substituents from the group consisting of halogen, alkyl, hydroxy, alkoxy, amino, alkylamino, dialkylamino, hydroxycarbonyl, alkoxycarbonyl, alkylcarbonylamino and alkoxycarbonylamino,
and
n represents an integer in the range from 1 to 10 inclusive,
   wherein in the case that more than one group X is present their individual meanings can be identical or different,
to give a peptide or protein conjugate of the formula (I)

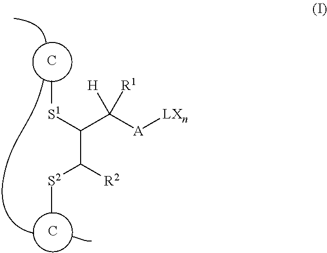

in which R¹, R², A, L, X and n have the meanings given above.

The process according to the invention is summarized in the reaction scheme below:

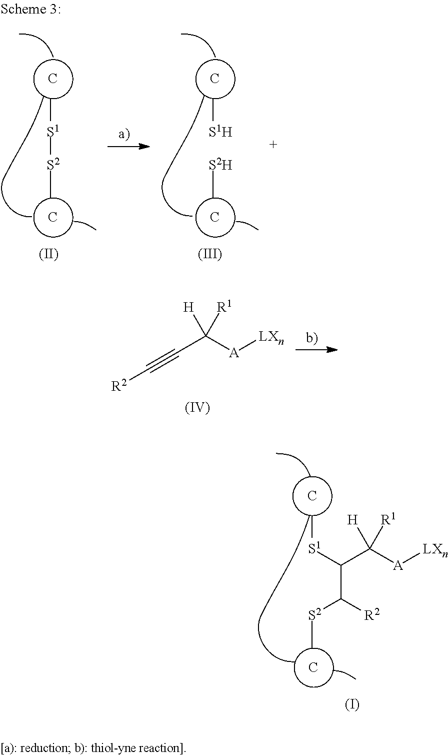

The reaction steps a) and b) shown in Scheme 3 can be carried out either separately, with intermediate isolation of the intermediate (III), or in succession in the same reaction vessel. Preferably, the reactions are carried out by the latter “one-pot process”. The reduction of the disulphide (II) to the free dithiol (III) is preferably carried out using tris(2-carboxyethyl)phosphine (TCEP). The thiol-yne reaction of the dithiol (III) with the alkyne derivative of the formula (IV) to the conjugate of the formula (I), which proceeds via a free-radical mechanism, can be mediated by photochemical free-radical initiators or oxidatively generated radicals such as, for example, triethylborane with small amounts of oxygen. Preference is given to using known photochemical free-radical initiators such as, for example, bis(2,4,6-trimethylbenzoyl)phenylphosphine oxide (Irgacure® 819) or lithium phenyl-2,4,6-trimethylbenzoylphosphinate (LAP) [Gong et al., 2013]. UV light is suitable for the photochemical initiation in combination with the free-radical initiator. Preference is given to UV light having a wavelength between 350 nm and 400 nm; particular preference is given to the wavelengths of 365 nm and 385 nm. Preferred inert solvents for the reaction steps a) and b) are water, aqueous buffer solutions or mixtures of water with a water-soluble organic solvent such as methanol or ethanol. The reactions are generally carried out in a temperature range from 0° C. to 40° C., preferably at room temperature.

If the peptide or protein to be conjugated according to the invention is already present in the dithiol form (III), the reduction step a) listed above does not apply; in this case, the process according to the invention relates to the “direct” reaction of the dithiol (III) with the alkyne derivative (IV) to give the conjugate of the formula (I) under the conditions described above for reaction step b).

The process according to the invention also comprises the preparation of multiple conjugates of a peptide or protein of the formula (II) with the alkyne derivative (IV) according to the reaction sequence described above in cases where in the peptide or protein of the formula (II) a plurality of disulphide bridges accessible to such a conjugation are present.

The present invention furthermore provides peptide and protein conjugates of the general formula (I)

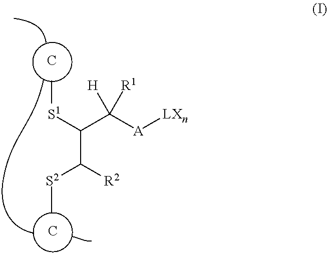

in which
S¹ and S² represent cysteine sulphur atoms, previously bonded in a disulphide bridge, of a peptide or protein,
R¹ and R² independently of one another represent hydrogen, alkyl, cycloalkyl, heterocycloalkyl, aryl, heteroaryl, hydroxy, alkoxy, amino, alkylamino, dialkylamino, hydroxycarbonyl, alkoxycarbonyl, alkylcarbonylamino or alkoxycarbonylamino,
   where alkyl, cycloalkyl, heterocycloalkyl, aryl, heteroaryl, alkoxy, alkylamino, dialkylamino, alkoxycarbonyl, alkylcarbonylamino and alkoxycarbonylamino for their part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino,
A represents a bond or a hydrocarbon chain having 1 to 100 carbon atoms from alkylene, cycloalkylene and/or arylene groups which may be interrupted once or more than once by identical or different groups selected from the group consisting of —O—, —S—, —S(═O)—, —S(═O)₂—, —NH—, —N(CH₃)—, —C(═O)—, —NH—C(═O)—, —C(═O)—NH—, —O—C(═O)—, —C(═O)—O—, —SO₂—NH—, —NH—SO₂—, —NH—NH—, —SO₂—NH—NH—, —NH—NH—SO₂—, —C(═O)—NH—NH—, —NH—NH—C(═O)—, —NH—C(═O)—NH—, —O—C(═O)—NH—, —NH—C(═O)—O— and a 4- to 10-membered aromatic or non-aromatic heterocycle having up to 4 heteroatoms from the group consisting of N, O, S, S(═O) and S(═O)₂,
or
R² and A are attached to one another and together with the interjacent carbon atoms form an 8-membered carbocycle which may be fused to a 3- to 6-membered cycloalkyl ring,
   where the 8-membered carbocycle and optionally the fused cycloalkyl ring may be mono- or polysubstituted by identical or different substituents from the group consisting of fluorine, alkyl, hydroxy, hydroxyalkyl and alkoxy,
L represents a bond or a linker,
X may be present n-times and represents an active compound molecule, a polymer, an alkaloid, a peptide, a protein, a carbohydrate, a nucleotide, a nucleoside, a steroid, a terpene, a porphyrin, a chlorin, a corrin, an eicosanoid, a pheromone, a vitamin, a biotin, a dye molecule or a cryptand or represents hydrogen, hydroxy, alkoxy, amino, alkylamino, dialkylamino, hydroxycarbonyl, alkoxycarbonyl, alkylcarbonylamino, alkoxycarbonylamino, alkyl, cycloalkyl, heterocycloalkyl, aryl or heteroaryl,
   where alkyl for its part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino
   and
   where cycloalkyl, heterocycloalkyl, aryl and heteroaryl for their part may be mono- or polysubstituted by identical or different substituents from the group consisting of halogen, alkyl, hydroxy, alkoxy, amino, alkylamino, dialkylamino, hydroxycarbonyl, alkoxycarbonyl, alkylcarbonylamino and alkoxycarbonylamino,
and
n represents an integer in the range from 1 to 10 inclusive,
   wherein in the case that more than one group X is present their individual meanings can be identical or different.

The present invention encompasses, in the case of peptides or proteins having a plurality of disulphide bridges accessible to the conjugation method according to the invention, also corresponding multiple conjugates of such a peptide or protein, i.e. conjugates where a pairwise C2-bridging in the sense of the formula (I) has taken place in a plurality of positions of the precursor peptide or protein in question.

In the context of the present invention, unless specified otherwise, the substituents and radicals are defined as follows: In the context of the invention, alkyl represents a straight-chain or branched alkyl radical having 1 to 10, preferably 1 to 8, particularly preferably 1 to 6, carbon atoms. The following may be mentioned by way of example and by way of preference: methyl, ethyl, n-propyl, isopropyl, n-butyl, isobutyl, sec-butyl, tert-butyl, n-pentyl, isopentyl, 1-ethylpropyl, 1-methylbutyl, 2-methylbutyl, 3-methylbutyl, neopentyl, n-hexyl, 1-methylpentyl, 2-methylpentyl, 3-methylpentyl, 4-methylpentyl, 3,3-dimethylbutyl, 1-ethylbutyl, 2-ethylbutyl, n-heptyl, n-octyl, n-nonyl and n-decyl.

In the context of the invention, alkylene represents a straight-chain or branched divalent alkyl radical (alkanediyl radical) having 1 to 10, preferably 1 to 8, particularly preferably 1 to 6, carbon atoms. The following may be mentioned by way of example and by way of preference: methylene, ethane-1,1-diyl, ethane-1,2-diyl (1,2-ethylene), propane-1,1-diyl, propane-1,2-diyl, propane-2,2-diyl, propane-1,3-diyl (1,3-propylene), butane-1,2-diyl, butane-1,3-diyl, butane-2,3-diyl, butane-1,4-diyl (1,4-butylene), pentane-1,5-diyl (1,5-pentylene), hexane-1,6-diyl (1,6-hexylene), heptane-1,7-diyl (1,7-heptylene), octane-1,8-diyl (1,8-octylene), nonane-1,9-diyl (1,9-nonylene) and decane-1,10-diyl (1,10-decylene).

In the context of the invention, hydroxyalkyl represents a straight-chain or branched alkyl radical having 1 to 6, preferably 1 to 4, carbon atoms which carries a hydroxyl group as a substituent in the chain or in a terminal position. The following may be mentioned by way of example and by way of preference: hydroxymethyl, 1-hydroxyethyl, 2-hydroxyethyl, 1-hydroxy-1-methylethyl, 1,1-dimethyl-2-hydroxyethyl, 1-hydroxypropyl, 2-hydroxypropyl, 3-hydroxypropyl, 1-hydroxy-2-methylpropyl, 2-hydroxy-1-methylpropyl, 2-hydroxy-2-methylpropyl, 1-hydroxybutyl, 2-hydroxybutyl, 3-hydroxybutyl, 4-hydroxybutyl, 5-hydroxypentyl and 6-hydroxyhexyl.

In the context of the invention, alkoxy represents a straight-chain or branched alkoxy radical having 1 to 6, preferably 1 to 4, carbon atoms. The following may be mentioned by way of example and by way of preference: methoxy, ethoxy, n-propoxy, isopropoxy, n-butoxy, isobutoxy, sec-butoxy, tert-butoxy, n-pentoxy, isopentoxy, 1-ethylpropoxy, 1-methylbutoxy, 2-methylbutoxy, 3-methylbutoxy and n-hexoxy.

In the context of the invention, alkoxycarbonyl represents a straight-chain or branched alkoxy radical which has 1 to 6, preferably 1 to 4, carbon atoms and is attached to the remainder of the molecule via a carbonyl group [—C(═O)—] bonded to the oxygen atom. The following may be mentioned by way of example and by way of preference: methoxycarbonyl, ethoxycarbonyl, n-propoxycarbonyl, isopropoxycarbonyl, n-butoxycarbonyl and tert-butoxycarbonyl.

In the context of the invention, alkylamino represents an amino group having a straight-chain or branched alkyl substituent having 1 to 6, preferably 1 to 4, carbon atoms. The following may be mentioned by way of example and by way of preference: methylamino, ethylamino, n-propylamino, isopropylamino, n-butylamino and tert-butylamino.

In the context of the invention, dialkylamino represents an amino group having two identical or different straight-chain or branched alkyl substituents having in each case 1 to 6, preferably 1 to 4, carbon atoms. The following may be mentioned by way of example and by way of preference: N,N-dimethylamino, N,N-diethylamino, N-ethyl-N-methylamino, N-methyl-N-n-propylamino, N-isopropyl-N-methylamino, N-isopropyl-N-n-propylamino, N,N-diisopropylamino, N-n-butyl-N-methylamino and N-tert-butyl-N-methylamino.

In the context of the invention, alkoxycarbonylamino represents an amino group with a straight-chain or branched alkoxycarbonyl substituent which has 1 to 6, preferably 1 to 4, carbon atoms in the alkoxy radical and is attached to the nitrogen atom via the carbonyl group. The following may be mentioned by way of example and by way of preference: methoxycarbonylamino, ethoxycarbonylamino, n-propoxycarbonylamino, isopropoxycarbonylamino, n-butoxycarbonylamino and tert-butoxycarbonylamino.

In the context of the invention, alkylcarbonyl represents a straight-chain or branched alkyl radical which has 1 to 6, preferably 1 to 4, carbon atoms and is attached to the remainder of the molecule via a carbonyl group [—C(═O)—]. The following may be mentioned by way of example and by way of preference: acetyl, propionyl, n-butyryl, isobutyryl, n-pentanoyl and pivaloyl.

In the context of the invention, alkylcarbonylamino represents an amino group with a straight-chain or branched alkylcarbonyl substituent which has 1 to 6, preferably 1 to 4, carbon atoms in the alkyl radical and is attached to the nitrogen atom via the carbonyl group. The following may be mentioned by way of example and by way of preference: acetylamino, propionylamino, n-butyrylamino, isobutyrylamino, n-pentanoylamino and pivaloylamino.

In the context of the invention, cycloalkyl represents a monocyclic saturated carbocycle having 3 to 10, preferably 3 to 8, particularly preferably 3 to 6, ring carbon atoms. The following may be mentioned by way of example and by way of preference: cyclopropyl, cyclobutyl, cyclopentyl, cyclohexyl, cycloheptyl, cyclooctyl, cyclononyl and cyclodecyl.

In the context of the invention, cycloalkylene represents a monocyclic saturated divalent cycloalkyl radical (cycloalkanediyl radical) having 3 to 10, preferably 3 to 8, particularly preferably 3 to 6, ring carbon atoms. The following may be mentioned by way of example and by way of preference: cyclopropane-1,1-diyl, cyclopropane-1,2-diyl, cyclobutane-1,1-diyl, cyclobutane-1,2-diyl, cyclobutane-1,3-diyl, cyclopentane-1,1-diyl, cyclopentane-1,2-diyl, cyclopentane-1,3-diyl, cyclohexane-1,1-diyl, cyclohexane-1,2-diyl, cyclohexane-1,3-diyl, cyclohexane-1,4-diyl, cycloheptane-1,1-diyl, cycloheptane-1,2-diyl, cycloheptane-1,4-diyl, cyclooctane-1,2-diyl, cyclooctane-1,5-diyl, cyclononane-1,2-diyl, cyclononane-1,5-diyl, cyclodecane-1,2-diyl und cyclodecane-1,6-diyl.

In the context of the invention, heterocycloalkyl represents a 4- to 10-membered mono- or optionally bicyclic non-aromatic heterocycle which is saturated or contains a double bond and has a total of 4 to 10 ring atoms, which contains up to four ring heteroatoms from the group consisting of N, O, S, S(═O) and/or S(═O)₂ and is attached via a ring carbon atom or optionally a ring nitrogen atom. The following may be mentioned by way of example: azetidinyl, oxetanyl, thietanyl, pyrrolidinyl, pyrrolinyl, pyrazolidinyl, dihydropyrazolyl, tetrahydrofuranyl, thiolanyl, 1,1-dioxidothiolanyl, 1,3-oxazolidinyl, 1,3-thiazolidinyl, piperidinyl, tetrahydropyridyl, piperazinyl, tetrahydropyranyl, dihydropyranyl, tetrahydrothiopyranyl, 1,3-dioxanyl, 1,4-dioxanyl, morpholinyl, thiomorpholinyl, 1,1-dioxidothiomorpholinyl, hexahydroazepinyl, hexahydro-1,4-diazepinyl, octahydroazocinyl, octahydropyrrolo[3,4-b]pyrrolyl, octahydroisoindolyl, octahydropyrrolo[3,4-b]pyridyl, hexahydropyrrolo[3,4-c]pyridyl, octahydropyrrolo[1,2-a]pyrazinyl, decahydroisoquinolinyl, octahydropyrido[1,2-a]pyrazinyl, 7-azabicyclo [2.2.1]heptanyl, 3-azabicyclo[3.2.0]heptanyl, 3-azabicyclo[3.2.1]octanyl, 8-azabicyclo[3.2.1]octanyl and 8-oxa-3-azabicyclo[3.2.1]octanyl. Preference is given to a 4- to 6-membered monocyclic saturated heterocycle which has a total of 4 to 6 ring atoms, which contains one or two ring heteroatoms from the group consisting of N, O and S and is attached via a ring carbon atom or optionally a ring nitrogen atom. The following may be mentioned by way of example: azetidinyl, oxetanyl, thietanyl, pyrrolidinyl, pyrazolidinyl, tetrahydrofuranyl, thiolanyl, 1,3-oxazolidinyl, piperidinyl, piperazinyl, tetrahydropyranyl, tetrahydrothiopyranyl, 1,4-dioxanyl, morpholinyl and thiomorpholinyl.

In the context of the invention, aryl represents an aromatic carbocycle having 6 or 10 ring carbon atoms, such as phenyl and naphthyl.

In the context of the invention, arylene represents a divalent aryl radical such as, for example, 1,2-phenylene, 1,3-phenylene, 1,4-phenylene, naphthalene-2,3-diyl, naphthalene-1,4-diyl, naphthalene-1,5-diyl, naphthalene-2,6-diyl and naphthalene-1,8-diyl.

In the context of the invention, heteroaryl represents a 5- to 10-membered monocyclic or optionally bicyclic aromatic heterocycle (heteroaromatic) which has a total of 5 to 10 ring atoms, contains up to four ring heteroatoms from the group of N, O and S and is joined via a ring carbon atom or optionally a ring nitrogen atom. The following may be mentioned by way of example: furyl, pyrrolyl, thienyl, pyrazolyl, imidazolyl, thiazolyl, oxazolyl, isoxazolyl, isothiazolyl, triazolyl, oxadiazolyl, thiadiazolyl, tetrazolyl, pyridyl, pyrimidinyl, pyridazinyl, pyrazinyl, triazinyl, benzofuranyl, benzothienyl, benzimidazolyl, benzoxazolyl, benzothiazolyl, benzotriazolyl, indolyl, indazolyl, quinolinyl, isoquinolinyl, naphthyridinyl, quinazolinyl, quinoxalinyl, phthalazinyl and pyrazolo[3,4-b]pyridinyl. Preference is given to a 5- or 6-membered monocyclic heteroaryl radical which has a total of 5 or 6 ring atoms, which contains up to three ring heteroatoms from the group consisting of N, O and S and is attached via a ring carbon atom or optionally a ring nitrogen atom. The following may be mentioned by way of example: furyl, thienyl, thiazolyl, oxazolyl, isothiazolyl, isoxazolyl, pyrazolyl, imidazolyl, triazolyl, oxadiazolyl, thiadiazolyl, pyridyl, pyrimidinyl, pyridazinyl, pyrazinyl and triazinyl.

Halogen in the context of the invention includes fluorine, chlorine, bromine and iodine. Preference is given to chlorine, fluorine or bromine, particular preference to fluorine or chlorine.

In the context of the present invention, all radicals which occur more than once are defined independently of one another. When radicals in the compounds according to the invention are substituted, the radicals may be mono- or polysubstituted, unless specified otherwise. Substitution by one or by two or three identical or different substituents is preferred. Substitution by one or by two identical or different substituents is particularly preferred. Very particular preference is given to substitution by one substituent.

For the purpose of the present invention, a “linker” represents a hydrocarbon chain having 1 to 100 carbon atoms from alkylene, cycloalkylene and/or arylene groups which may be interrupted once or more than once by identical or different groups selected from the group consisting of —O—, —S—, —S(═O)—, —S(═O)₂—, —NH—, —N(CH₃)—, —C(═O)—, —NH—C(═O)—, —C(═O)—NH—, —O—C(═O)—, —C(═O)—O—, —SO₂—NH—, —NH—SO₂—, —NH—NH—, —SO₂—NH—NH—, —NH—NH—SO₂—, —C(═O)—NH—NH—, —NH—NH—C(═O)—, —NH—C(═O)—NH—, —O—C(═O)—NH—, —NH—C(═O)—O— and a 4- to 10-membered aromatic or non-aromatic heterocycle having up to 4 heteroatoms from the group consisting of N, O, S, S(═O) and S(═O)₂ and by an in vivo cleavable group or by an in vivo transiently stable group.

The term “in vivo cleavable group” can be subdivided into groups which can be cleaved by chemical means in vivo (for example by acid hydrolysis or redox processes) and those which can be cleaved enzymatically, i.e. via action of an endogenous enzyme, in vivo. Both types of cleavable groups should initially be stable in the circulatory system and only be cleaved at or in the target cell by the different chemical or enzymatic environment (e.g. lower pH, increased glutathione concentration, presence of lysosomal enzymes such as cathepsin or plasmin) at that location. Chemically in vivo cleavable structural fragments suitable for this purpose are in particular disulphide, hydrazone, acetal and aminal groupings. Enzymatically in vivo cleavable structural elements are in particular oligopeptide units of 2 to 8 amino acids, and here in particular dipeptide groupings. Numerous of such designated peptide cleavage sites have been described in the literature. Prominent examples are the dipeptide units valine-alanine, valine-lysine, valine-citrulline, alanine-lysine and phenylalanine-lysine [see, for example, J. J. Petersen and C. F. Meares, Bioconjugate Chem. 9, 618-626 (1998); G. M. Dubowchik and R. A. Firestone, Bioorg. Med. Chem. Lett. 8, 3341-3346 (1998); G. M. Dubowchik et al., Bioconjugate Chem. 13, 855-869 (2002)].

The linker described above may also contain an in vivo transiently stable group. Such transiently stable groups are cleaved under the action of the chemical or enzymatic environment, for example in the circulatory system, over a prolonged period of time (of hours or days) [see, for example, Flamme et al., Int. Pat. Appl. WO 2013/064455-A1].

Suitable active compound molecules in the above definition of group X are in particular pharmaceuticals for cancer therapy such as cytotoxins and cytostatics. In detail, these active compounds are capable of damaging the cancer cell, initiating apoptosis and/or suppressing cell growth and cell proliferation. As examples of such active compounds for cancer therapy, the following may be mentioned: antimetabolites such as methotrexate, cladribine, fludarabine, mercaptopurine, tioguanine, pentostatin, cytarabine, fluorouracil, capecitabine and gemcitabine, alkylating agents such as cyclophosphamide, ifosfamide, mitomycin, trofosfamide, thiotepa, busulfan, treosulfan, carmustine, lomustine, nimustine, procarbazine, dacarbazine and the platinum compounds cisplatin, carboplatin and oxaliplatin, topoisomerase inhibitors such as topotecan, irinotecan, etoposide and teniposide, kinase inhibitors such as sorafenib, regorafinib, sunitinib, afatinib, erlotinib and gefitinib, mitose inhibitors such as vinblastine, vincristine, vinorelbine, paclitaxel and docetaxel, and also antibiotics such as dactinomycin, daunorubicin, doxorubicin, idarubicin, epirubicin, bleomycin, mitoxantrone and amsacrine.

Further active compounds which are included in the scope of group X are cytotoxic compounds and toxins, such as those which have been investigated experimentally and clinically as “toxophores” in antibody drug conjugates (ADCs) in particular for cancer therapy. Examples which may be mentioned here are in particular substances such as maytansine and maytansinoids (DM-1, DM-4), dolastatins, auristatins (MMAE, MMAF), calicheamicins, duocarmycins, camptothecins (topotecan, exatecan, irinotecan, SN-38), doxorubicin, amatoxins (amanitin), pyrrolobenzodiazepines (PBDs) and kinesin spindle protein (KSP) inhibitors.

According to the present invention, the peptides and proteins of the formula (II) contain at least two cysteine amino acids forming a disulphide bond or capable of forming a disulphide bond. Such peptides include, for example, peptide hormones such as insulin, somatostatin, oxitocin, terlipressin, adrenomedullin, calcitonin or vasopressin. Examples of proteins of this type which may be mentioned are antibodies, cytokines such as interleukins and also albumins.

In accordance with the present invention, the term “antibody” is to be understood in its broadest meaning and refers to immunoglobulin molecules, for example intact or modified monoclonal antibodies, polyclonal antibodies or multispecific antibodies (e.g. bispecific antibodies), and also fragments thereof. An immunoglobulin molecule preferably represents a molecule having four polypeptide chains, consisting of two heavy chains (H chains, HC) and two light chains (L chains, LC), which are typically attached to one another via disulphide bridges (interchain disulphide bridges). Each heavy chain comprises a variable domain (abbreviated V_{H}) and a constant domain (C_{H}). For its part, the constant domain of the heavy chain may have three (C_{H}1, C_{H}2, C_{H}3) or four subdomains. Each light chain also comprises a variable domain (V_{L}) and a constant domain (C_{L}). The V_{H} and V_{L} domains may be subdivided further into regions having hypervariability, also referred to as complementarity determining regions (CDRs), and regions having lower sequence variability (framework region, FR). Each V_{H} and V_{L} region is typically composed of three CDRs and up to four FRs, for example from the amino to the carboxyl terminus in the sequence FR1, CDR1, FR2, CDR2, FR3, CDR3, FR4. An antibody may be obtained from any suitable species, e.g. monkey, pig, rabbit, mouse or rat. In a particular embodiment, the antibody is of human or murine origin. Such an antibody may, for example, be human, humanized or chimeric.

The present invention furthermore provides for the use of the conjugates of the formula (I) for the diagnosis and/or treatment of disorders, in particular for the diagnosis and/or treatment of cancer and tumour disorders.

The present invention further provides for the use of the conjugates of the formula (I) in a method for the diagnosis and/or treatment of disorders, in particular of cancer and tumour disorders.

The present invention further provides a method for the diagnosis and/or treatment of disorders, in particular of cancer and tumour disorders, using one or more conjugates of the formula (I).

For the purpose of the present invention, the term “treatment” or “treating” includes inhibition, retardation, checking, alleviating, attenuating, restricting, reducing, suppressing, repelling or healing of a disease, a condition, a disorder, an injury or a health problem, or the development, the course or the progression of such states and/or the symptoms of such states. The term “therapy” is understood here to be synonymous with the term “treatment”.

In the context of the present invention, the term “diagnosis” is understood in the usual sense as the (differentiating) identification, recognition, determination, assessment, classification and naming of a disease, a condition, a disorder, a disease symptom, an injury or a health problem.

The present invention further provides pharmaceutical compositions which comprise at least one of the conjugates of the formula (I), typically together with one or more inert, nontoxic, pharmaceutically suitable auxiliaries, and for the use thereof for the aforementioned purposes.

The conjugates of the formula (I) according to the invention can act systemically and/or locally. For this purpose, they can be administered in a suitable manner, for example by the oral, parenteral, pulmonal, nasal, sublingual, lingual, buccal, rectal, dermal, transdermal, conjunctival or otic route, or as an implant or stent.

The conjugates according to the invention can be administered in administration forms suitable for these administration routes.

Suitable administration forms for oral administration are those which work according to the prior art and release the conjugates according to the invention rapidly and/or in a modified manner and which contain the conjugates according to the invention in crystalline and/or amorphized and/or dissolved form, for example tablets (uncoated or coated tablets, for example with gastric juice-resistant or retarded-dissolution or insoluble coatings which control the release of the conjugates according to the invention), tablets or films/oblates which disintegrate rapidly in the oral cavity, films/lyophilizates, capsules (for example hard or soft gelatin capsules), sugar-coated tablets, granules, pellets, powders, emulsions, suspensions, aerosols or solutions.

Parenteral administration can be accomplished with avoidance of a resorption step (for example by an intravenous, intraarterial, intracardiac, intraspinal or intralumbar route) or with inclusion of a resorption (for example by an intramuscular, subcutaneous, intracutaneous, percutaneous or intraperitoneal route). Administration forms suitable for parenteral administration include preparations for injection and infusion in the form of solutions, suspensions, emulsions, lyophilizates or sterile powders.

For the other administration routes, suitable examples are inhalation medicaments (including powder inhalers, nebulizers), nasal drops, solutions or sprays; tablets for lingual, sublingual or buccal administration, films/oblates or capsules, suppositories, ear or eye preparations, vaginal capsules, aqueous suspensions (lotions, shaking mixtures), lipophilic suspensions, ointments, creams, transdermal therapeutic systems (e.g. patches), milk, pastes, foams, dusting powders, implants or stents.

Preference is given to parenteral administration, especially intravenous administration.

The conjugates according to the invention can be converted to the administration forms mentioned. This can be accomplished in a manner known per se by mixing with inert, non-toxic, pharmaceutically suitable excipients. These excipients include carriers (for example microcrystalline cellulose, lactose, mannitol), solvents (e.g. liquid polyethylene glycols), emulsifiers and dispersing or wetting agents (for example sodium dodecylsulphate, polyoxysorbitan oleate), binders (for example polyvinylpyrrolidone), synthetic and natural polymers (for example albumin), stabilizers (e.g. antioxidants, for example ascorbic acid), colorants (e.g. inorganic pigments, for example iron oxides) and flavour and/or odour correctants.

The working examples which follow illustrate the invention. The invention is not restricted to the examples.

### A. EXAMPLES

### Abbreviations and Acronyms

abs. absolute, of absolute purity
aq. aqueous, aqueous solution
Boc tert-butoxycarbonyl
c concentration
DMF N,N-dimethylformamide
DMPA 2,2-dimethoxy-2-phenylacetophenone
DMSO dimethyl sulphoxide
DPBS Dulbecco's phosphate buffered saline
DTT dithiothreitol
ELSD evaporative light scattering detector
eq. equivalent(s)
ES or ESI electrospray ionization (in MS)
FAB fragment antigen-binding
h hour(s)
HPLC high-pressure, high-performance liquid chromatography
HRMS high-resolution mass spectrometry
Irgacure® 819 bis(2,4,6-trimethylbenzoyl)phenylphosphine oxide
conc. concentrated (in the case of a solution)
LAP lithium phenyl-2,4,6-trimethylbenzoylphosphinate
LC/MS liquid chromatography-coupled mass spectrometry
LED light-emitting diode
lit. literature (reference)
m multiplet (in NMR)
m/z mass/charge ratio (in MS)
min minute(s)
MPLC medium-pressure liquid chromatography (on silica gel; also referred to as flash chromatography)
MS mass spectrometry
NMR nuclear magnetic resonance spectrometry
PBS phosphate-buffered saline
RP reverse phase (in HPLC)
RT room temperature
Rₜ retention time (in HPLC, LC/MS)
TCEP-HCl tris(2-carboxyethyl)phosphine hydrochloride
tert tertiary
TFA trifluoroacetic acid
THF tetrahydrofuran
TOF time-of-flight mass spectrometry
UV ultraviolet (spectrometry)
v/v ratio by volume (of a solution)

LC/MS methods:

Method 1:

Instrument: Waters Acquity SQD UPLC System; column: Waters Acquity UPLC HSS T3 1.8 μm, 50×1 mm; mobile phase A: 1 l of water+0.25 ml of 99% strength formic acid, mobile phase B: 1 l of acetonitrile+0.25 ml of 99% strength formic acid; gradient: 0.0 min 95% A→6.0 min 5% A→7.5 min 5% A; oven: 50° C.; flow rate: 0.35 ml/min; UV detection: 210-400 nm.

Method 2:

MS instrument type: Waters Synapt G2S; UPLC instrument type: Waters Acquity I-Class; column: Waters HSS T3, 2.1×50 mm, C18 1.8 μm; mobile phase A: 1 l of water+0.01% formic acid, mobile phase B: 1 l of acetonitrile+0.01% formic acid; gradient: 0.0 min 2% B→2.0 min 2% B→13.0 min 90% B→15.0 min 90% B; oven: 50° C.; flow rate: 1.20 ml/min; UV detection: 210 nm.

Method 3:

MS instrument type: Thermo Fisher Scientific LTQ-Orbitrap-XL; HPLC instrument type: Agilent 1200SL; column: Agilent Poroshell 120 SB-C18 2.7 μm, 3×150 mm; mobile phase A: 1 l of water+0.1% trifluoroacetic acid, mobile phase B: 1 l of acetonitrile+0.1% trifluoroacetic acid; gradient: 0.0 min 2% B→1.5 min 2% B→15.5 min 95% B→18.0 min 95% B; oven: 40° C.; flow rate: 0.75 ml/min; UV detection: 210 nm.

Starting Compounds:

Compound A

### N-(2,5,8,11,14,17,20,23-Octaoxapentacosan-25-yl)hex-5-ynamide

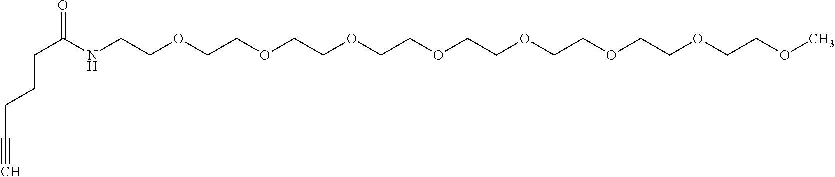 [SMILES: C#CCCCC(=O)NCCOCCOCCOCCOCCOCCOCCOCCOC]

Under an atmosphere of argon, 27.8 μl (0.25 mmol) of hex-5-ynoic acid and 48.3 mg (0.25 mmol) of 1,1′-carbonyldiimidazole were initially charged in 1 ml of absolute DMF. The mixture was stirred at RT for 2 h. 100.0 mg (0.25 mmol) of 2,5,8,11,14,17,20,23-octaoxapentacosane-25-amine, dissolved in 0.5 ml of abs. DMF, were then added. The mixture was stirred at RT overnight and then concentrated under reduced pressure. The residue was purified by preparative MPLC. The product-containing fractions were concentrated, 10 ml of water were added to the residue and the mixture was extracted three times with in each case 10 ml of ethyl acetate. The combined organic phases were dried over magnesium sulphate and concentrated under reduced pressure, and the residue was dried under high vacuum (product yield: 22 mg). The aqueous phase obtained beforehand was lyophilized and the lyophilizate was purified by silica gel chromatography (mobile phase: first dichloromethane/methanol 100:5, then dichloromethane/methanol 9:1). This gave 37.8 mg of a transparent oil.

Total yield: 59.8 mg (51% of theory)

¹H-NMR (400 MHz, CDCl₃): [ppm]=1.87 (quin, J=7.1 Hz, 2H), 1.99 (t, J=2.5 Hz, 1H), 2.26 (td, J=6.9 and 2.7 Hz, 2H), 2.32 (t, J=7.4 Hz, 2H), 3.38 (s, 3H), 3.45 (q, J=5.4 Hz, 2H), 3.55 (m, 4H), 3.60-3.71 (m, 26H), 6.18 (br. s, 1H).

¹³C-NMR (125.78 MHz, CDCl₃): δ [ppm]=172.21, 83.61, 71.93, 70.60, 70.56, 70.53, 70.51, 70.25, 69.87, 69.12, 59.03, 39.18, 35.00, 24.18, 17.88.

Compound B

### 4-(Hex-5-yn-1-yl)-4-methylmorpholin-4-ium iodide

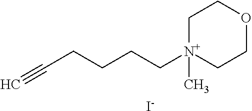

3.0 ml (22.7 mmol) of 6-iodohex-1-yne were added dropwise to 2.5 ml (22.7 mmol) of 4-methylmorpholine. The mixture was stirred at RT for 5 minutes and then at 54° C. for 10 minutes. The mixture was then cooled to RT and stirred for another 16 h, and small amounts of petroleum ether, diethyl ether and finally ethyl acetate were then added. The precipitate was filtered off and the filtrate was concentrated under reduced pressure. The resulting precipitate was filtered off and washed with ethyl acetate. The white to beige-coloured powder was dried under high vacuum.

Yield: 593.6 mg (9% of theory).

¹H-NMR (400 MHz, D₂O): [ppm]=1.62 (quin, J=7.3 Hz, 2H), 1.95 (m, 2H), 2.33 (td, J=7 and 2.6 Hz, 2H), 2.41 (t, J=2.6 Hz, 1H), 3.21 (s, 3H), 3.46-3.60 (m, 6H), 4.06 (br. s, 4H).

¹³C-NMR (125.78 MHz, D₂O): δ [ppm]=84.64, 70.01, 66.59, 60.42, 59.64, 59.58, 24.23, 20.13, 17.11.

MS (ESpos): m/z=182.2.

### WORKING EXAMPLES

### Example 1

### (5R,12R)-12-{[(Benzyloxy)carbonyl]amino}-5-carboxy-8-(8-carboxyoctyl)-3-oxo-1-phenyl-2-oxa-7,10-dithia-4-azatridecan-13-oic acid

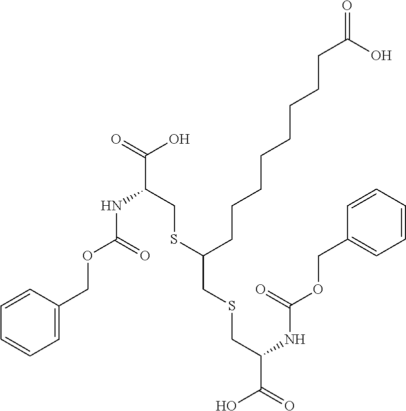 [SMILES: O=C(O)CCCCCCCCC(CSC[C@H](NC(=O)OCc1ccccc1)C(=O)O)SC[C@H](NC(=O)OCc1ccccc1)C(=O)O]

In a two-necked round-bottom flask, 100.00 mg (0.19 mmol) of N,N′-bis[(benzyloxy)carbonyl]-L-cystine and 79.47 mg (277.25 μmol) of TCEP-HCl were initially charged in 1 ml of water/methanol (1:1 v/v) under an atmosphere of argon. The mixture was stirred at RT for 2.5 h. 33.69 mg (0.19 mmol) of undec-10-ynoic acid and 3.87 mg (9.24 μmol) of Irgacure® 819 were then added. Via a ground-glass joint of the two-necked flask, an LED-UV head (OmniCure LX400, diameter 12 mm; igb-tech GmbH, Germany) was introduced (distance to the reaction mixture about 40 mm), and the reaction mixture was irradiated with 365 nm UV light for 1 h. Conversion according to HPLC (mobile phase: gradient acetonitrile/water+0.1% TFA; ELSD) was quantitative. The reaction solution was then diluted with methanol and purified by preparative HPLC (mobile phase: gradient acetonitrile/water+0.1% TFA). This gave 39 mg (27% of theory, purity according to LC/MS 88%) of the target compound.

LC/MS (Method 1): Rₜ=3.45 min; MS (ESIpos): m/z=693 [M+H]⁺

¹H-NMR (400 MHz, DMSO-d₆): δ [ppm]=1.24-1.50 (m, 13H), 1.55-1.60 (m, 2H), 1.72-1.79 (m, 1H), 2.66-3.18 (m, 7H), 4.34-4.40 (m, 2H), 5.07-5.14 (m, 4H), 7.26-7.38 (m, 10H).

### Examples 2A and 2B

### 2A: 5-[(6R,9S,12S,15S,18S,21R)-21-{[({[(Aminoacetyl)amino]acetyl}amino)acetyl]amino}-6-{[(2S)-2-({(2S)-6-amino-1-[(2-amino-2-oxoethyl)amino]-1-oxohexan-2-yl}carbamoyl)pyrrolidin-1-yl]carbonyl}-9-(2-amino-2-oxoethyl)-12-(3-amino-3-oxopropyl)-15-benzyl-18-(4-hydroxybenzyl)-8,11,14,17,20-pentaoxo-1,4-dithia-7,10,13,16,19-pentaazacyclodocosan-3-yl]pentanoic acid

### 2B: 5-[(6R,9S,12S,15S,18S,21R)-21-{[({[(Aminoacetyl)amino]acetyl}amino)acetyl]amino}-6-{[(2S)-2-({(2S)-6-amino-1-[(2-amino-2-oxoethyl)amino]-1-oxohexan-2-yl}carbamoyl)pyrrolidin-1-yl]carbonyl}-9-(2-amino-2-oxoethyl)-12-(3-amino-3-oxopropyl)-15-benzyl-18-(4-hydroxybenzyl)-8,11,14,17,20-pentaoxo-1,4-dithia-7,10,13,16,19-pentaazacyclodocosan-2-yl]pentanoic acid

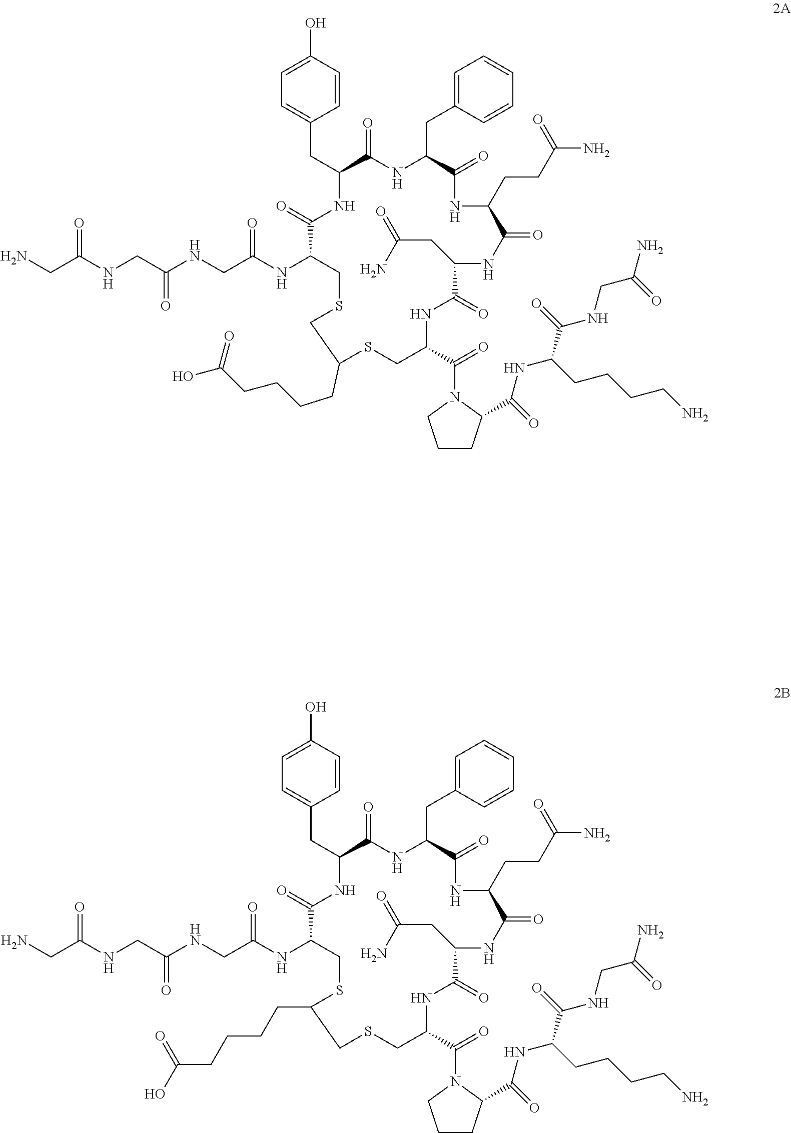 [SMILES: NCCCC[C@H](NC(=O)[C@@H]1CCCN1C(=O)[C@@H]1CSC(CCCCC(=O)O)CSC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O ; NCCCC[C@H](NC(=O)[C@@H]1CCCN1C(=O)[C@@H]1CSCC(CCCCC(=O)O)SC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O]

Under an atmosphere of argon, 50.17 mg (37.23 μmol) of terlipressin acetate (from Bachem, Switzerland; sequence: H-Gly-Gly-Gly-Cys-Tyr-Phe-Gln-Asn-Cys-Pro-Lys-Gly-NH₂, as cyclic Cys disulphide) were initially charged in a two-necked flask in 1.1 ml of water, and 16.01 mg (55.85 μmol) of TCEP-HCl were added. The reaction mixture was stirred at RT for 2 h, and a solution of 4.70 mg (37.23 mmol) of hept-6-ynoic acid in 100 μl of methanol was then added. 0.55 mg (1.86 μmol) of LAP [prepared by a process known from the literature (Gong et al., 2013)] in 150 μl of water was then added. An LED-UV head (OmniCure LX400, diameter 12 mm; igb-tech GmbH, Germany) was introduced via a ground-glass joint of the two-necked flask (distance to the reaction mixture about 40 mm), and the reaction mixture was irradiated with 365 nm UV light for 1 h. Another 0.55 mg (1.86 μmol) of LAP was then added, and the reaction mixture was irradiated with 365 nm UV light for another 1 h. The reaction mixture was then purified by separating the isomeric products by preparative HPLC (column: Waters X-Bridge BEH130 Prep C18 10 μm OBD, 19×250 mm; mobile phase A: water with 0.05% TFA, mobile phase B: acetonitrile with 0.05% TFA; gradient: 0.0 min 5% B→40 min 40% B).

Isomer 1:

LC/MS (Method 2): Rₜ=3.53 min; MS (ESpos): m/z=1355 [M+H]⁺.

Isomer 2:

Yield: 1.3 mg (2.6% of theory)

LC/MS (Method 2): Rₜ=3.57 min; MS (ESpos): m/z=1355 [M+H]⁺

¹³C-NMR (125.78 MHz, D₂O, δ (1,4-dioxane)=67.4 ppm): δ [ppm]=183.1 (S), 182.1 (S), 178.5 (S), 175.2 (3S), 174.7 (S), 174.0 (S), 173.9 (S), 172.7 (S), 172.5 (S), 171.5 (S), 170.9, 168.7, 155.2 (S), 137.0 (S), 131.4 (S), 130.0 (S), 129.7 (S), 128.8 (S), 128.2 (S), 116.3 (S), 61.7 (D), 57.1 (D), 55.6 (D), 55.3 (D), 54.5 (D), 53.4 (D), 52.6 (D), 50.7 (D), 48.8 (T), 45.7 (D), 43.2 (T), 43.0 (T), 42.9 (T), 41.3 (T), 40.1 (T), 38.8 (T), 37.0 (T), 36.6 (T), 36.4 (2T), 34.0 (T), 33.6 (T), 31.9 (2T), 30.9 (T), 30.1 (T), 27.0 (T), 26.9 (T), 26.8 (T), 25.6 (T), 25.5 (T), 22.9 (T) [corresponds to a ring-closed structure since no olefinic carbon atoms can be detected].

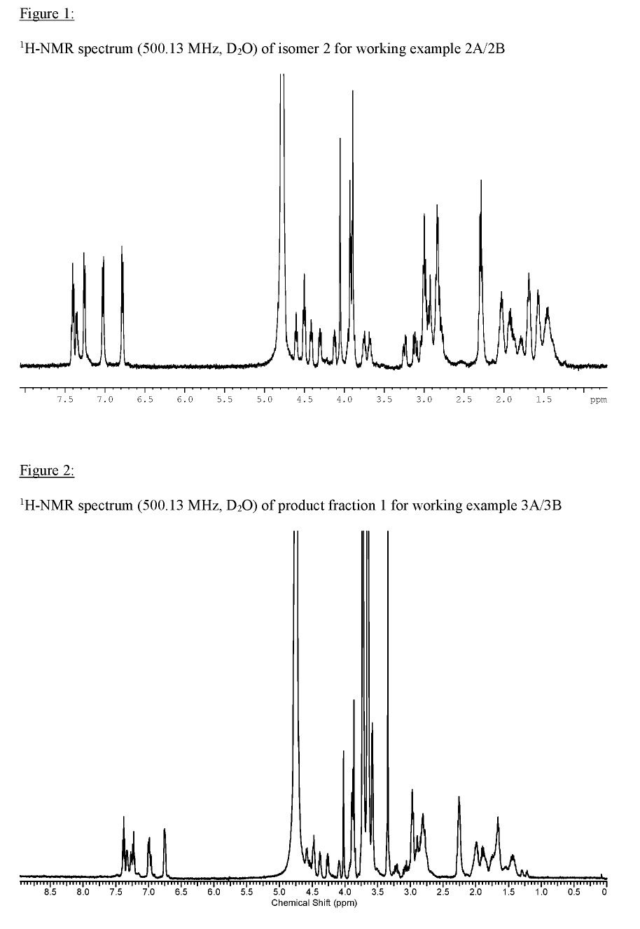

The ¹H NMR spectrum of this isomer is shown in FIG. 1.

### Examples 3A and 3B

### 3A: (2S)-1-{[(6R,9S,12S,15S,18S,21R)-21-{[({[(Aminoacetyl)amino]acetyl}amino)acetyl]amino}-9-(2-amino-2-oxoethyl)-12-(3-amino-3-oxopropyl)-15-benzyl-18-(4-hydroxybenzyl)-8,11,14,17,20-pentaoxo-3-(27-oxo-2,5,8,11,14,17,20,23-octaoxa-26-azatriacontan-30-yl)-1,4-dithia-7,10,13,16,19-pentaazacyclodocosan-6-yl]carbonyl}-N-{(2S)-6-amino-1-[(2-amino-2-oxoethyl)amino]-1-oxohexan-2-yl}pyrrolidine-2-carboxamide

### 3B: (2S)-1-{[(6R,9S,12S,15S,18S,21R)-21-{[({[(Aminoacetyl)amino]acetyl}amino)acetyl]amino}-9-(2-amino-2-oxoethyl)-12-(3-amino-3-oxopropyl)-15-benzyl-18-(4-hydroxybenzyl)-8,11,14,17,20-pentaoxo-2-(27-oxo-2,5,8,11,14,17,20,23-octaoxa-26-azatriacontan-30-yl)-1,4-dithia-7,10,13,16,19-pentaazacyclodocosan-6-yl]carbonyl}-N-{(2S)-6-amino-1-[(2-amino-2-oxoethyl)amino]-1-oxohexan-2-yl}pyrrolidine-2-carboxamide

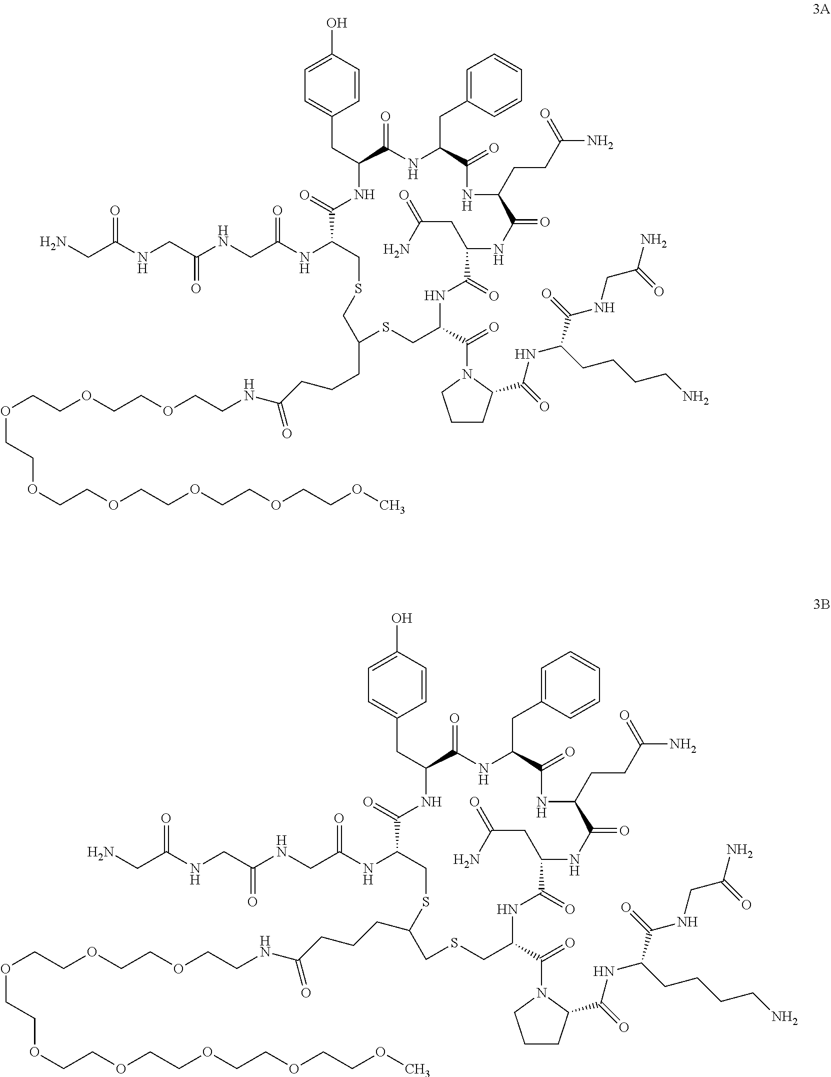 [SMILES: COCCOCCOCCOCCOCCOCCOCCOCCNC(=O)CCCC1CSC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N[C@H](C(=O)N2CCC[C@H]2C(=O)N[C@@H](CCCCN)C(=O)NCC(N)=O)CS1 ; COCCOCCOCCOCCOCCOCCOCCOCCNC(=O)CCCC1CSC[C@@H](C(=O)N2CCC[C@H]2C(=O)N[C@@H](CCCCN)C(=O)NCC(N)=O)NC(=O)[C@H](CC(N)=O)NC(=O)[C@H](CCC(N)=O)NC(=O)[C@H](Cc2ccccc2)NC(=O)[C@H](Cc2ccc(O)cc2)NC(=O)[C@@H](NC(=O)CNC(=O)CNC(=O)CN)CS1]

Under an atmosphere of argon, 20.64 mg (15.32 μmol) of terlipressin acetate (from Bachem, Switzerland; sequence: H-Gly-Gly-Gly-Cys-Tyr-Phe-Gln-Asn-Cys-Pro-Lys-Gly-NH₂, as cyclic Cys disulphide) were initially charged in a two-necked flask in 500 μl of DPBS buffer, and 13.30 mg (46.39 mol) of TCEP-HCl were added. The reaction mixture was stirred at RT for 2 h, and a solution of 7.20 mg (15.15 μmol) of N-(2,5,8,11,14,17,20,23-octaoxapentacosan-25-yl)hex-5-ynamide in 150 μl of DPBS buffer was then added. 4.4 mg (14.95 μmol) of LAP [prepared by a process known from the literature (Gong et al., 2013)] were then dissolved in 500 μl of DPBS buffer, and 50 μl of this LAP solution were then added to the reaction mixture. An LED-UV head (OmniCure LX400, diameter 12 mm; igb-tech GmbH, Germany) was introduced via a ground-glass joint of the two-necked flask (distance to the reaction mixture about 40 mm), and the reaction mixture was irradiated with 365 nm UV light for 1 h. The addition of 50 μl of the LAP solution and the subsequent irradiation with 365 nm UV light for 1 h were then repeated two more times. The reaction mixture was then fractionated by preparative HPLC (column: Waters X-Bridge BEH130 Prep C18 10 μm OBD, 19×250 mm; mobile phase A: water with 0.1% TFA, mobile phase B: acetonitrile with 0.1% TFA; gradient: 0.0 min 5% B→3 min 5% B→43 min 40% B→44.30 min 95% B→49.30 min 95% B).

Product Fraction 1:

Yield: 6.20 mg (23% of theory); purity according to LC/MS (Method 3): 99%

LC/MS (Method 3): Rₜ=7.41 min; MS (ESpos): m/z=853.9101 [M+2H]²⁺

HRMS: calculated for C₇₅H₁₂₁O₂₄N₁₇S₂ [M+2H]²⁺: 853.9100, measured: 853.9100.

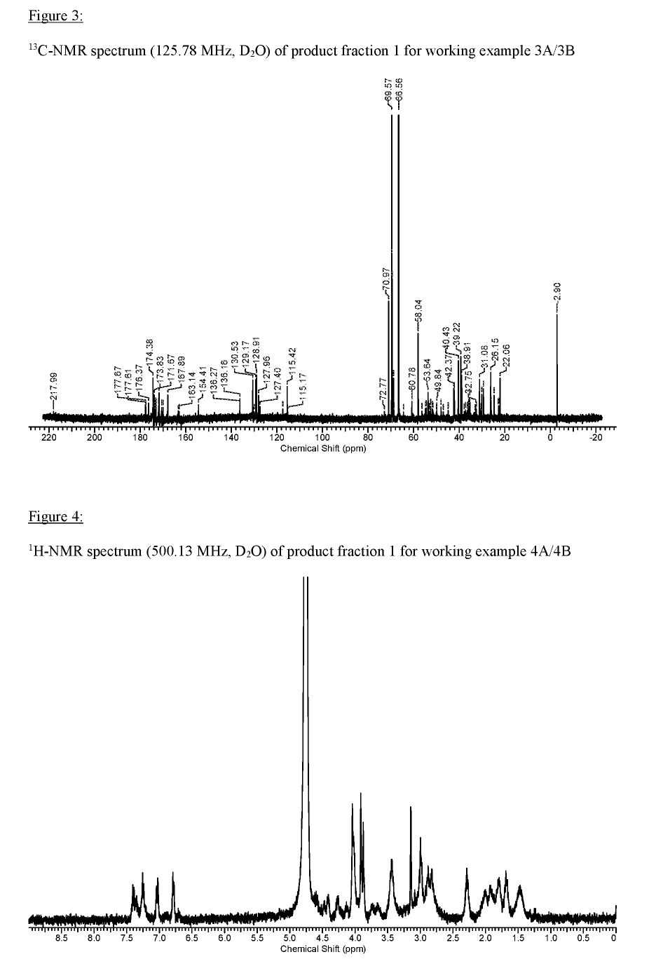

The ¹H and ¹³C NMR spectra of this product fraction are shown in FIGS. 2 and 3.

Product Fraction 2:

Yield: 1.70 mg (7% of theory)

LC/MS (Method 2): Rₜ=4.09 min; MS (ESpos): m/z=853.9149 [M+2H]²⁺

HRMS: calculated for C₇₅H₁₂₁O₂₄N₁₇S₂[M+2H]²⁺: 853.9100, measured: 853.9095.

### Examples 4A and 4B

### 4A: 4-{4-[(6R,9S,12S,15S,18S,21R)-21-{[({[(Aminoacetyl)amino]acetyl}amino)acetyl]amino}-6-{[(2S)-2-({(2S)-6-amino-1-[(2-amino-2-oxoethyl)amino]-1-oxohexan-2-yl}carbamoyl)pyrrolidin-1-yl]carbonyl}-9-(2-amino-2-oxoethyl)-12-(3-amino-3-oxopropyl)-15-benzyl-18-(4-hydroxybenzyl)-8,11,14,17,20-pentaoxo-1,4-dithia-7,10,13,16,19-pentaazacyclodocosan-3-yl]butyl}-4-methylmorpholin-4-iumn trifluoroacetate

### 4B: 4-{4-[(6R,9S 12S,15S,18S,21R)-21-{[({[(Aminoacetyl)amino]acetyl}amino)acetyl]amino}-6-{[(25)-2-({(2S)-6-amino-1-[(2-amino-2-oxoethyl)amino]-1-oxohexan-2-yl}carbamoyl)pyrrolidin-1-yl]carbonyl}-9-(2-amino-2-oxoethyl)-12-(3-amino-3-oxopropyl)-15-benzyl-18-(4-hydroxybenzyl)-8,11,14,17,20-pentaoxo-1,4-dithia-7,10,13,16,19-pentaazacyclodocosan-2-yl]butyl}-4-methylmorpholin-4-ium trifluoroacetate

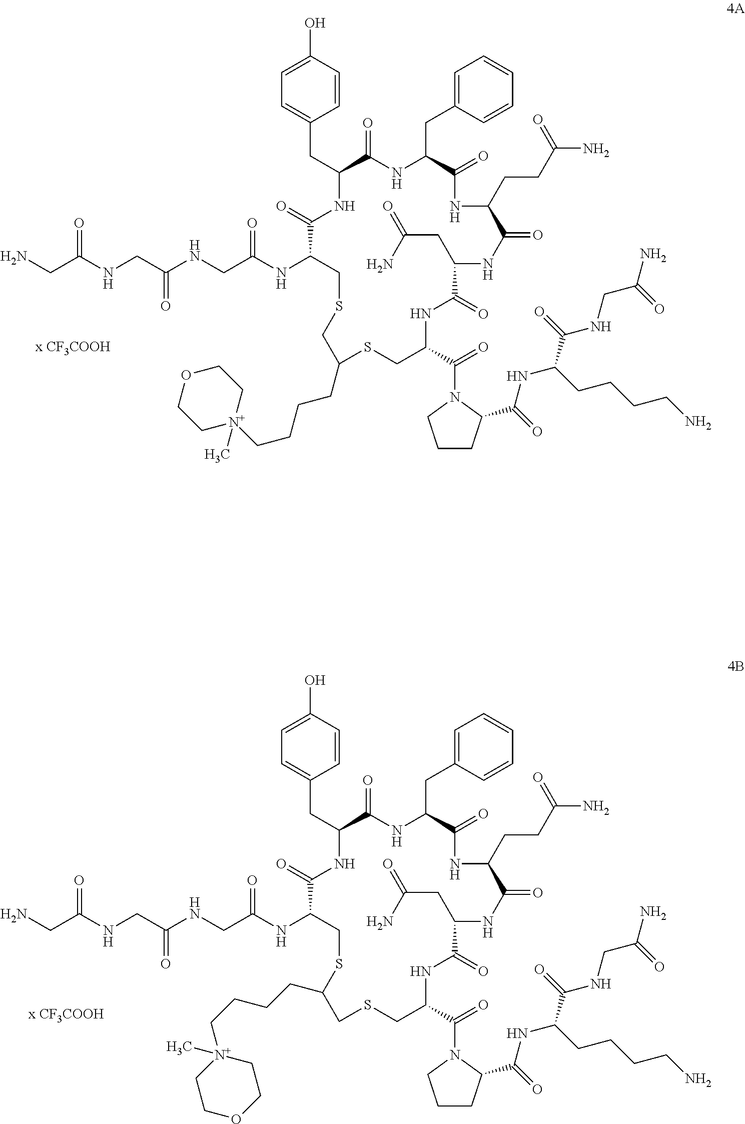 [SMILES: C[N+]1(CCCCC2CSC[C@H](NC(=O)CNC(=O)CNC(=O)CN)C(=O)N[C@@H](Cc3ccc(O)cc3)C(=O)N[C@@H](Cc3ccccc3)C(=O)N[C@@H](CCC(N)=O)C(=O)N[C@@H](CC(N)=O)C(=O)N[C@H](C(=O)N3CCC[C@H]3C(=O)N[C@@H](CCCCN)C(=O)NCC(N)=O)CS2)CCOCC1 ; C[N+]1(CCCCC2CSC[C@@H](C(=O)N3CCC[C@H]3C(=O)N[C@@H](CCCCN)C(=O)NCC(N)=O)NC(=O)[C@H](CC(N)=O)NC(=O)[C@H](CCC(N)=O)NC(=O)[C@H](Cc3ccccc3)NC(=O)[C@H](Cc3ccc(O)cc3)NC(=O)[C@@H](NC(=O)CNC(=O)CNC(=O)CN)CS2)CCOCC1]

Under an atmosphere of argon, 21.54 mg (15.98 mol) of terlipressin acetate (from Bachem, Switzerland; sequence: H-Gly-Gly-Gly-Cys-Tyr-Phe-Gln-Asn-Cys-Pro-Lys-Gly-NH₂, as cyclic Cys disulphide) were initially charged in a two-necked flask in 800 μl of DPBS buffer, and 6.50 mg (22.68 μmol) of TCEP-HCl were added. The reaction mixture was stirred at RT for 2 h, and a solution of 4.9 mg (15.84 μmol) of 4-(hex-5-yn-1-yl)-4-methylmorpholin-4-ium iodide in 500 μl of DPBS buffer was then added. 4.70 mg (15.98 μmol) of LAP [prepared by a process known from the literature (Gong et al., 2013)] were then dissolved in 500 μl of DPBS buffer, and 50 μl of this LAP solution were then added to the reaction mixture. An LED-UV head (OmniCure LX400, diameter 12 mm; igb-tech GmbH, Germany) was introduced via a ground-glass joint of the two-necked flask (distance to the reaction mixture about 40 mm), and the reaction mixture was irradiated with 365 nm UV light for 1 h. The addition of 50 μl of the LAP solution and the subsequent irradiation with 365 nm UV light for 1 h were then repeated two more times. The reaction mixture was then fractionated by preparative HPLC (column: Phenomenex Kinetex Prep 5 μm C18 100 Å AXIA Packed LC Column, 21.2×100 mm; mobile phase A: water with 0.1% TFA, mobile phase B: acetonitrile with 0.08% TFA; gradient: 0.0 min 5% B→3 min 5% B→63 min 40% B→64.30 min 95% B→69.30 min 95% B).

Product Fraction 1:

Yield: 1.5 mg (4% of theory); purity according to LC/MS (Method 3): 65%

LC/MS (Method 3): Rₜ=6.49 min; MS (ESpos): m/z=705.8397 [M+H]²⁺

HRMS: calculated for C₆₃H₉₇O₁₆N₁₇S₂ [M+H]²⁺: 705.8365, measured: 705.8361.

The ¹H NMR spectrum of this product fraction is shown in FIG. 4.

Product Fraction 2:

Yield: 2.2 ing (8% of theory); purity according to LC/MS (Method 3): 87%

LC/MS (Method 3): Rₜ=6.48 min; MS (ESpos): m/z=705.8401 [M+1H]²¹

HRMS: calculated for C₆₃H₉₇O₁₆N₁₇S₂[M+H]²⁺: 705.8365, measured: 705.8377.

The ¹H NMR spectrum of this product fraction is shown in FIG. 5.

### Examples 5A and 5B

### 5A: (2S)-3-[(6R,9S,12S,15S,18S,21R)-21-{[({[(Aminoacetyl)amino]acetyl}amino)acetyl]amino}-6-{[(2S)-2-({(2 S)-6-amino-1-[(2-amino-2-oxoethyl)amino]-1-oxohexan-2-yl}carbamoyl)pyrrolidi-1-yl]carbonyl}-9-(2-amino-2-oxoethyl)-12-(3-amino-3-oxopropyl)-15-benzyl-18-(4-hydroxybenzyl)-8,11,14,17,20-pentaoxo-1,4-dithia-7,10,13,16,19-pentaazacyclodocosan-3-yl]-2-[(tert-butoxycarbonyl)amino]propanoic acid

### 5B: (25)-3-[(6R,9S,12S,15S. 18S,21R)-21-{[({[(Aminoacetyl)amino]acetyl}amino)acetyl]amino}-6-{[(2S)-2-({(25)-6-amino-1-[(2-amino-2-oxoethyl)amino]-1-oxohexan-2-yl}carbamoyl)pyrrolidin-1-yl]carbonyl}-9-(2-amino-2-oxoethyl)-12-(3-amino-3-oxopropyl)-15-benzyl-18-(4-hydroxybenzyl)-8,11,14,17,20-pentaoxo-1,4-dithia-7,10,13,16,19-pentaazacyclodocosan-2-yl]-2-[(tert-butoxycarbonyl)amino]propanoic acid

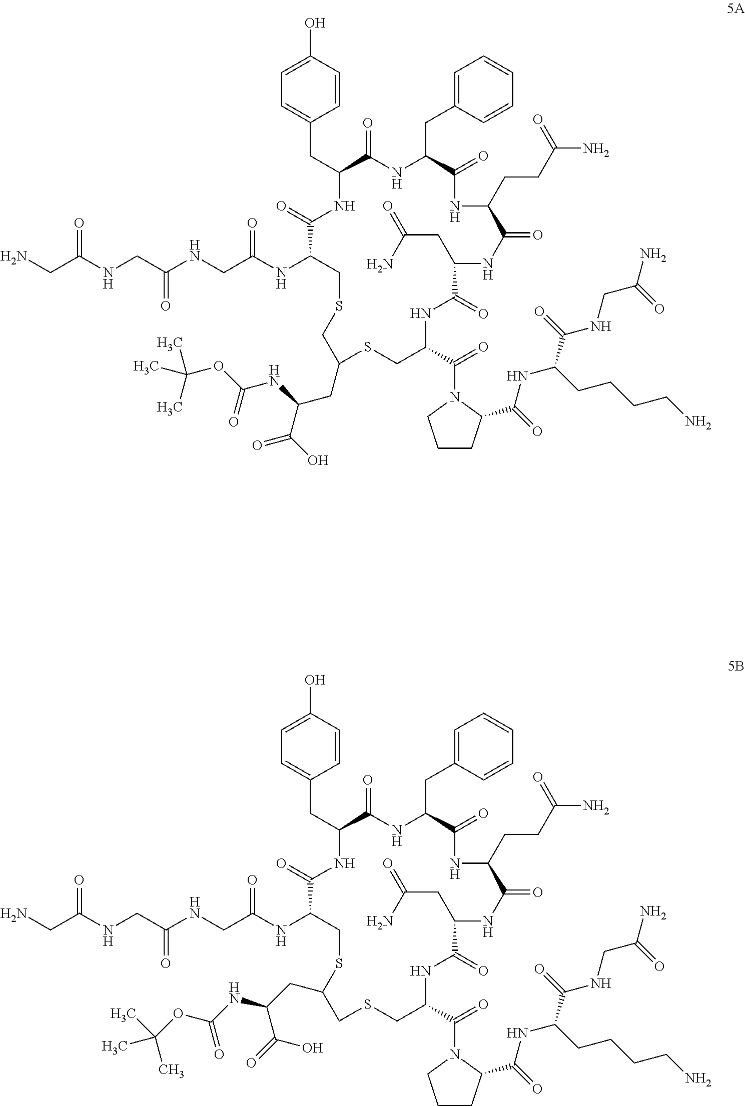 [SMILES: CC(C)(C)OC(=O)N[C@@H](CC1CSC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N[C@H](C(=O)N2CCC[C@H]2C(=O)N[C@@H](CCCCN)C(=O)NCC(N)=O)CS1)C(=O)O ; CC(C)(C)OC(=O)N[C@@H](CC1CSC[C@@H](C(=O)N2CCC[C@H]2C(=O)N[C@@H](CCCCN)C(=O)NCC(N)=O)NC(=O)[C@H](CC(N)=O)NC(=O)[C@H](CCC(N)=O)NC(=O)[C@H](Cc2ccccc2)NC(=O)[C@H](Cc2ccc(O)cc2)NC(=O)[C@@H](NC(=O)CNC(=O)CNC(=O)CN)CS1)C(=O)O]

Under an atmosphere of argon, 10.16 mg (7.54 μmol) of terlipressin acetate (from Bachern, Switzerland; sequence: H-Gly-Gly-Gly-Cys-Tyr-Phe-Gln-Asn-Cys-Pro-Lys-Gy-NH₂, as cyclic Cys disulphide) were initially charged in a two-necked flask in 300 μl of DPBS buffer, and 3.20 mg (11.17 μmol) of TCEP-HCl were added. The reaction mixture was stirred at RT for 1.5 h, and 200 μl of DPBS buffer and a solution of 1.6 mg (7.54 μmol) of N-Boc-L-propargylglycine in 100 μl of DPBS buffer were then added. 3.20 mg (10.87 μmol) of LAP [prepared by a process known from the literature (Gong et al., 2013)] were then dissolved in 500 μl of DPBS buffer, and 50 μl of this LAP solution were then added to the reaction mixture. An LED-UV head (OmniCure LX400, diameter 12 mm; igb-tech GmbH, Germany) was introduced via a ground-glass joint of the two-necked flask (distance to the reaction mixture about 40 mim), and the reaction mixture was irradiated with 365 nm UV light for 1 h. The addition of 50 μl of the LAP solution and the subsequent irradiation with 365 nm UV light for 1 h were then repeated two more times. The reaction mixture was then fractionated by preparative HPLC (column: Phenomenex Kinetex Prep 5 μm C18 100 Å AXIA Packed LC Column, 21.2×100 mm; mobile phase A: water with 0.1% TFA, mobile phase B: acetonitrile with 0.08% TFA; gradient: 0.0 min 5% B→3 min 5% B→63 min 40% B→65.3 min 95% B→70 min 95% B).

Product Fraction 1:

Yield: 0.4 mg (4% of theory)

LC/MS (Method 3): Rₜ=7.11 min; MS (ESpos): m/z=721.8130 [M+2H]²⁺

HRMS: calculated for C₆₂H₉₃O₁₉N₁₇S₂ [M+2H]²⁺: 721.8132, measured: 721.8130.

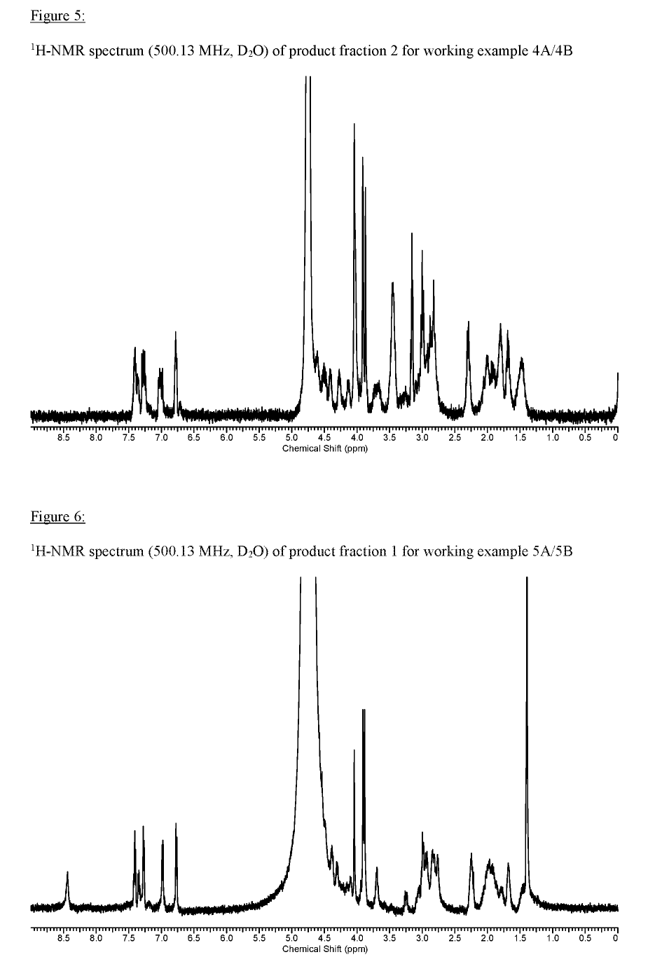

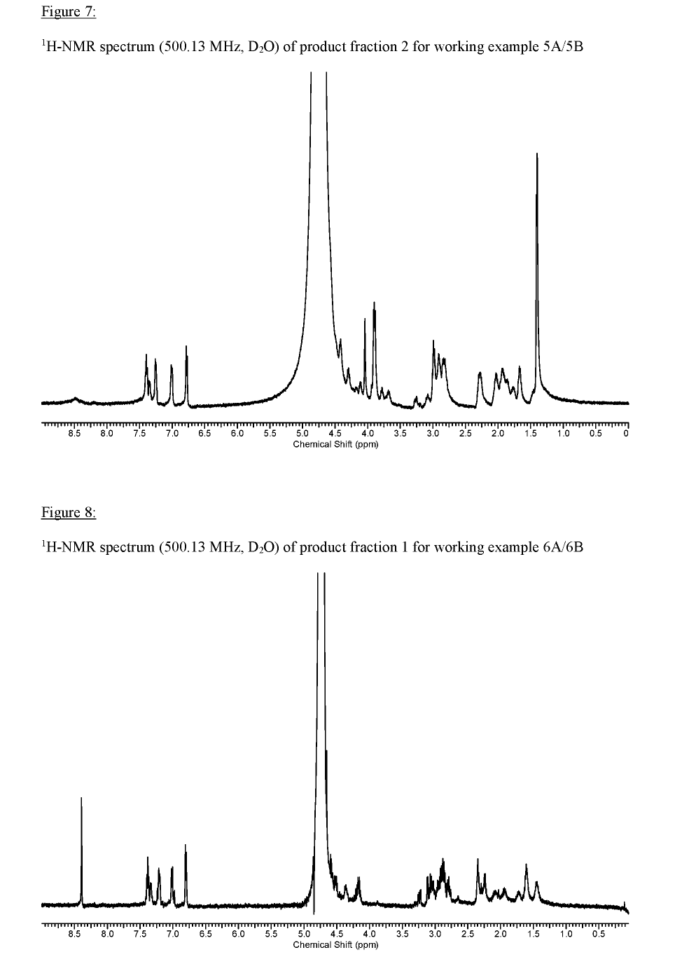

The ¹H NMR spectrum of this product fraction is shown in FIG. 6.

Product Fraction 2:

Yield: 1.6 mng (15% of theory); purity according to LC/MS (Method 3): 94%

LC/MS (Method 3): Rₜ=7.36 min; MS (ESpos): m/z=1442.6134 [M+H]⁺

HRMS: calculated for C₆₂H₉₃O₁₉N₁₇S₂[M+2H]²⁺: 721.8132, measured: 721.8132.

The ¹H NMR spectrum of this product fraction is shown in FIG. 7.

Product Fraction 3:

Yield: 0.3 mng (3% of theory); purity according to LC/MS (Method 3): 89%

LC/MS (Method 3): Rₜ=7.34 min; MS (ESpos): m/z=721.8122 [M+2H]²⁺

HRMS: calculated for C₆₂H₉₂O₁₉N₁₇S₂[M+H]⁺: 1442.6191, measured: 1442.6200.

### Examples 6A and 6B

### 6A: (6R,9S,12S,15S,18S,21R)-21-Amino-9-(2-amino-2-oxoethyl)-12-(3-amino-3-oxopropy)-15-benzyl-3-(4-carboxy butyl)-18-(4-hydroxy benzyl)-8,11,14,17,20-pentaoxo-1,4-dithia-7,10,13,16,19-pentaazacyclodocosane-6-carboxylic acid

### 6B: (6R,9S,12S,15S,18S,21R)-21-Amino-9-(2-amino-2-oxoethyl)-12-(3-amino-3-oxopropyl)-15-benzyl-2-(4-carboxybutyl)-18-(4-hydroxybenzyl)-8,11,14,17,20-pentaoxo-1,4-dithia-7,10,13,16,19-pentaazacyclodocosane-6-carboxylic acid

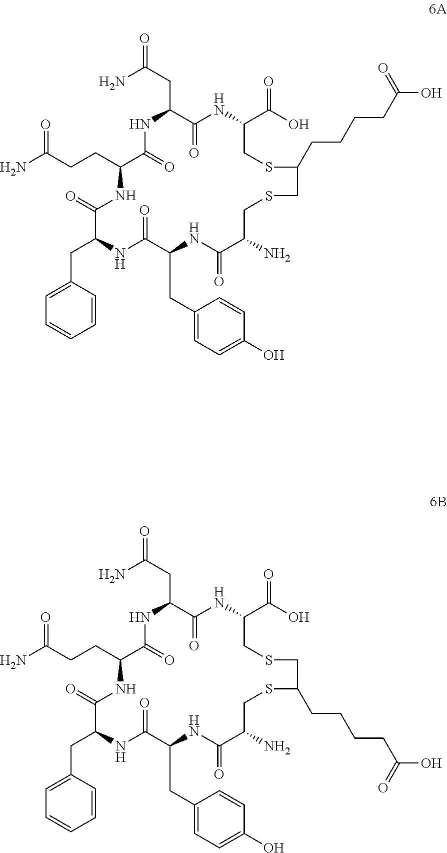 [SMILES: NC(=O)CC[C@@H]1NC(=O)[C@H](Cc2ccccc2)NC(=O)[C@H](Cc2ccc(O)cc2)NC(=O)[C@@H](N)CSCC(CCCCC(=O)O)SC[C@@H](C(=O)O)NC(=O)[C@H](CC(N)=O)NC1=O ; NC(=O)CC[C@@H]1NC(=O)[C@H](Cc2ccccc2)NC(=O)[C@H](Cc2ccc(O)cc2)NC(=O)[C@@H](N)CSC(CCCCC(=O)O)CSC[C@@H](C(=O)O)NC(=O)[C@H](CC(N)=O)NC1=O]

Under an atmosphere of argon, 15.33 mg (19.78 μmol) of pressinoic acid (from Bachem, Switzerland; sequence: H-Cys-Tyr-Phe-Gln-Asn-Cys-OH, as cyclic Cys disulphide) were initially charged in a two-necked flask in 500 μl of 0.1% strength aqueous acetic acid and 500 μl of acetonitrile, and 8.70 mg (30.35 mol) of TCEP-HCl were added. The reaction mixture was stirred at RT for 2.5 h, and 200 μl of 0.1% strength aqueous acetic acid, 200 μl of acetonitrile and a solution of 2.5 mg (19.82 μmol) of hept-6-ynoic acid in 100 μl of 0.1% strength aqueous acetic acid were then added. 5.8 mg (19.72 μmol) of LAP [prepared by a process known from the literature (Gong et al., 2013)] were then dissolved in 500 μl of 0.1% strength aqueous acetic acid, and 50 μl of this LAP solution were then added to the reaction mixture. An LED-UV head (OmniCure LX400, diameter 12 mm; igb-tech GmbH, Germany) was introduced via a ground-glass joint of the two-necked flask (distance to the reaction mixture about 40 mm), and the reaction mixture was irradiated with 365 nm UV light for 1 h. The addition of 50 μl of the LAP solution and the subsequent irradiation with 365 nm UV light for 1 h were then repeated two more times. The reaction mixture was then fractionated by preparative HPLC (column: Waters X-Bridge BEH130 Prep C18 10 μm OBD, 19×250 mm; mobile phase A: water with 0.1% TFA, mobile phase B: acetonitrile with 0.08% TFA; gradient: 0.0 min 5% B→3 min 5% B→33 min 40% B).

Product Fraction 1:

Yield: 1.4 mg (8% of theory)

LC/MS (Method 1): Rₜ=1.25 min; MS (ESpos): m/z=903.4 [M+H]⁺.

The ¹H NMR spectrum of this product fraction is shown in FIG. 8.

Product Fraction 2:

Yield: 0.6 mg (3% of theory)

LC/MS (Method 1): Rₜ=1.25 min; MS (ESpos): m/z=903.4 [M+H]⁺ and Rₜ=1.27 min; MS (ESpos): m/z=903.3 [M+H]⁺.

HRMS: calculated for C₄₀H₅₅O₁₂N₈S₂ [M+H]⁺: 903.3375 measured: 903.3371.

Product Fraction 3:

Yield: 0.7 mg (4% of theory)

LC/MS (Method 1): Rₜ=1.27 min; MS (ESpos): m/z=903.3 [M+H]⁺.

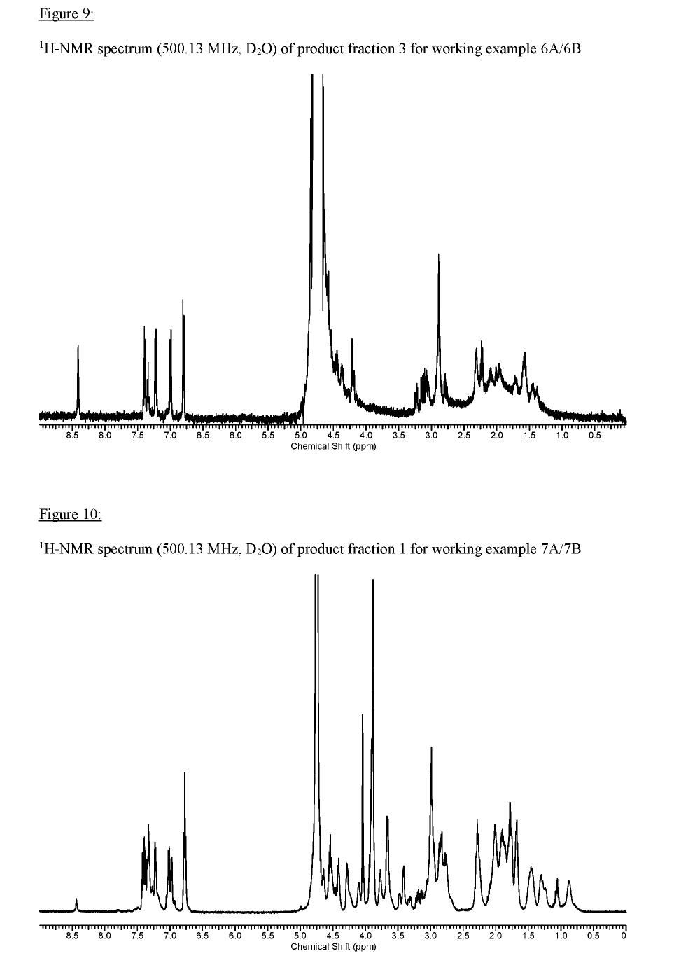

The ¹H NMR spectrum of this product fraction is shown in FIG. 9.

### Examples 7A and 7B

### 7A: (2S)-1-{[(3R,6S,9S,12S,15S,18R,22aR,23S,23aS)-18-{[({[(Aminoacetyl)amino]acetyl}amino)acetyl]amino}-6-(2-amino-2-oxoethyl)-9-(3-amino-3-oxopropyl)-12-benzyl-15-(4-hydroxybenzyl)-23-(hydroxymethyl)-5,8,11,14,17-pentaoxohexacosahydro-20aH-cyclopropa[5,6]cycloocta[1,2-b][1,4,7,10,13,16,19]dithiapentaazacyclodocosin-3-yl]carbonyl}-N-{(2S)-6-amino-1-[(2-amino-2-oxoethyl)amino]-1-oxohexan-2-yl}pyrrolidine-2-carboxamide

### 7B: (2S)-1-{[(3R,6S,9S,12S,15S,18R,22aS,23R,23aR)-18-{[({[(Aminoacetyl)amino]acetyl}amino)acetyl]amino}-6-(2-amino-2-oxoethyl)-9-(3-amino-3-oxopropyl)-12-benzyl-15-(4-hydroxybenzyl)-23-(hydroxymethyl)-5,8,11,14,17-pentaoxohexacosahydro-20aH-cyclopropa[5, 6]cycloocta[1,2-b][1,4,7,10,13,16,19]dithiapentaazacyclodocosin-3-yl]carbonyl}-N-{(2S)-6-amino-1-[(2-amino-2-oxoethyl)amino]-1-oxohexan-2-yl}pyrrolidine-2-carboxamide

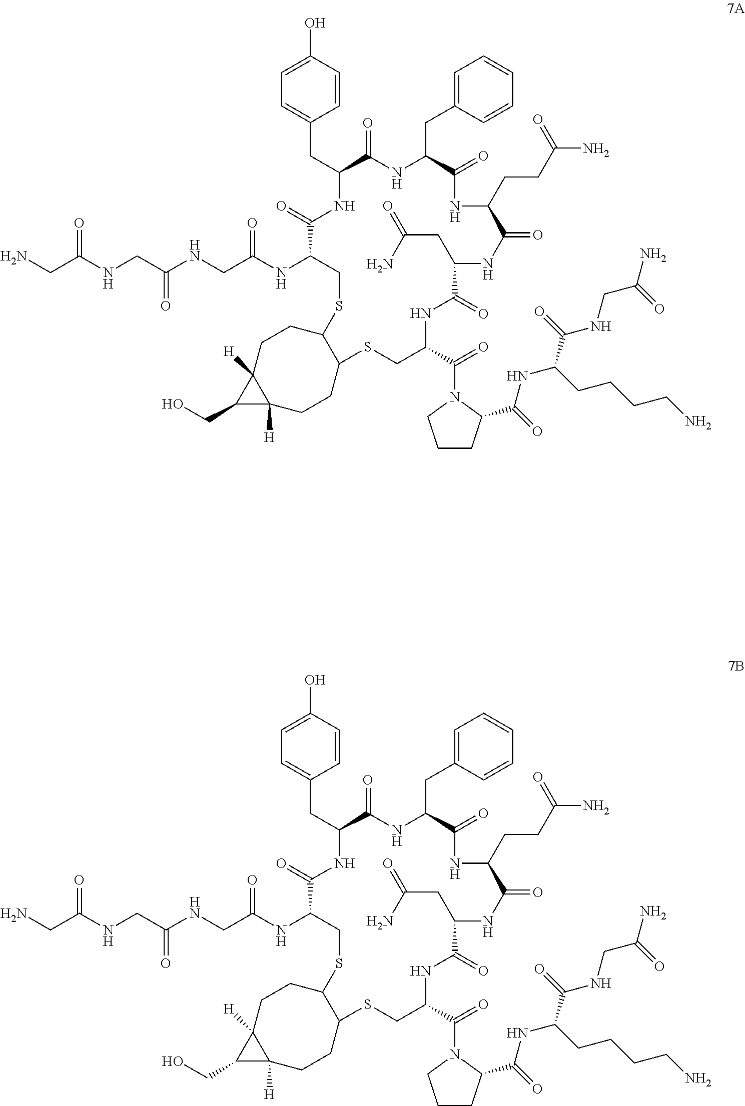 [SMILES: NCCCC[C@H](NC(=O)[C@@H]1CCCN1C(=O)[C@@H]1CSC2CC[C@@H]3[C@@H](CO)[C@@H]3CCC2SC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O ; NCCCC[C@H](NC(=O)[C@@H]1CCCN1C(=O)[C@@H]1CSC2CC[C@H]3[C@H](CO)[C@H]3CCC2SC[C@H](NC(=O)CNC(=O)CNC(=O)CN)C(=O)N[C@@H](Cc2ccc(O)cc2)C(=O)N[C@@H](Cc2ccccc2)C(=O)N[C@@H](CCC(N)=O)C(=O)N[C@@H](CC(N)=O)C(=O)N1)C(=O)NCC(N)=O]

Under an atmosphere of argon, 26.24 mg (19.47 μmol) of terlipressin acetate (from Bachem, Switzerland; sequence: H-Gly-Gly-Gly-Cys-Tyr-Phe-Gln-Asn-Cys-Pro-Lys-Gly-NH₂, as cyclic Cys disulphide) and 8.4 mg (29.31 μmol) of TCEP-HCl were initially charged in a two-necked flask in 1.5 ml of DPBS buffer. The reaction mixture was stirred at RT for 2.5 h, and a solution of 2.9 mg (19.32 μmol) of rel-(1R,8S,9r)-bicyclo[6.1.0]non-4-yn-9-ylmethanol in 200 μl of methanol was then added. 5.70 mg (19.38 μmol) of LAP [prepared by a process known from the literature (Gong et al., 2013)] were then dissolved in 500 μl of DPBS buffer, and 50 μl of this LAP solution were then added to the reaction mixture. An LED-UV head (OmniCure LX400, diameter 12 mm; igb-tech GmbH, Germany) was introduced via a ground-glass joint of the two-necked flask (distance to the reaction mixture about 40 mm), and the reaction mixture was irradiated with 365 nm UV light for 1 h. The addition of 50 μl of the LAP solution and the subsequent irradiation with 365 nm UV light for 1 h were then repeated two more times. The reaction mixture was then fractionated by preparative HPLC (column: Phenomenex Kinetex Prep 5 μm C18 100 Å AXIA Packed LC Column, 21.2×100 mm; mobile phase A: water with 0.1% TFA, mobile phase B: acetonitrile with 0.08% TFA; gradient: 0.0 min 5% B→3 min 5% B→63 min 40% B→64.30 min 95% B→69.30 min 95% B).

Product Fraction 1:

Yield: 3.1 mg (7.5% of theory); purity according to LC/MS (Method 3): 65%

LC/MS (Method 3): R₁=6.92 min; MS (ESpos): m/z=903.3 [M+H]⁺

HRMS: calculated for C₆₂H₉₁O₁₆N₁₆S₂[M+H]⁺: 1379.6235, measured: 1379.6244.

The ¹H NMR spectrum of this product fraction is shown in FIG. 10.

### Example 8

C2-Bridging Thiol-Yne Reaction with an Antibody FAB Fragment:

Under an atmosphere of argon, 108.4 μl of a solution of the FAB fragment M14-G07 [described in Dittmer et al., US Pat. Appl. US 2014/0050743-A1, page 13, sections 0105, 0106 and 0113ff.] were initially charged in PBS buffer (c=46.1 mg/ml, 0.107 μmol) in a two-necked flask. 4.60 mg of TCEP-HCl were dissolved in 400 μl of DPBS buffer, and 4 μl of this solution were added to the solution of the FAB fragment. The reaction mixture was stirred at RT for 1 h, and 4 μl of a solution of 1.36 μl (10.75 μmol) of hept-6-ynoic acid in 398 μl of methanol were then added. 3.10 mg (10.53 μmol) of LAP [prepared by a process known from the literature (Gong et al., 2013)] were then dissolved in 500 μl of DPBS buffer, and 1 μl of this LAP solution was then added to the reaction mixture. The reaction flask was cooled using an ice bath (5° C.<T<10° C.). An LED-UV head (OmniCure LX400, diameter 12 mm; igb-tech GmbH, Germany) was introduced via a ground-glass joint of the two-necked flask (distance to the reaction mixture about 40 mm), and the reaction mixture was irradiated with 365 nm UV light for 1 h. In three successive intervals, in each case another 1 μl of the LAP solution was added and the reaction mixture was subsequently irradiated with 365 nm UV light, in each case for 1 h. The reaction mixture was then diluted with 2.384 ml of DPBS buffer and fractionated on a Sephadex® G-25M PD-10 column (GE Healthcare) which had been conditioned five times with in each case 5 ml of DPBS buffer beforehand. The resulting fractions were centrifuged for 5 minutes (10° C., 4000 rpm). The solution was then pipetted into centrifuge filtration vessels (Ultracel® 30K—Amicon® Ultra-4) and centrifuged for another 15 minutes. The resulting concentrate was diluted repeatedly with a total of 2.5 ml of DPBS buffer.

Confirmation and Quantification of the Covalently Attached FAB Fragment:

From the resulting samples in DPBS buffer, the covalent attachment of the FAB fragment was quantified and identified as follows:

Quantification of the covalent FAB fragment was by RP chromatography of the reduced and denatured FAB fragment. Guanidinium hydrochloride (GuHCl) (28.6 mg) and a solution of DL-dithiothreitol (DTT) (500 mM, 3 μl) were added to the sample solution (1 mg/ml, 50 μl). The mixture was incubated at 55° C. for one hour and then analyzed by HPLC.

HPLC analysis was carried out on an Agilent 1260 HPLC system with detection at 220 nm. A Polymer Laboratories PLRP-S polymeric reversed-phase column (2.1 mm×150 mm, 8 μm particle size, 1000 Å, catalogue no. PL1912-3802) was used at a flow rate of 1 ml/min with the following mobile phase system: mobile phase A: 0.05% trifluoroacetic acid in water, mobile phase B: 0.05% trifluoroacetic acid in acetonitrile; gradient: 0 min 25% B, 3 min 25% B, 28 min 50% B.

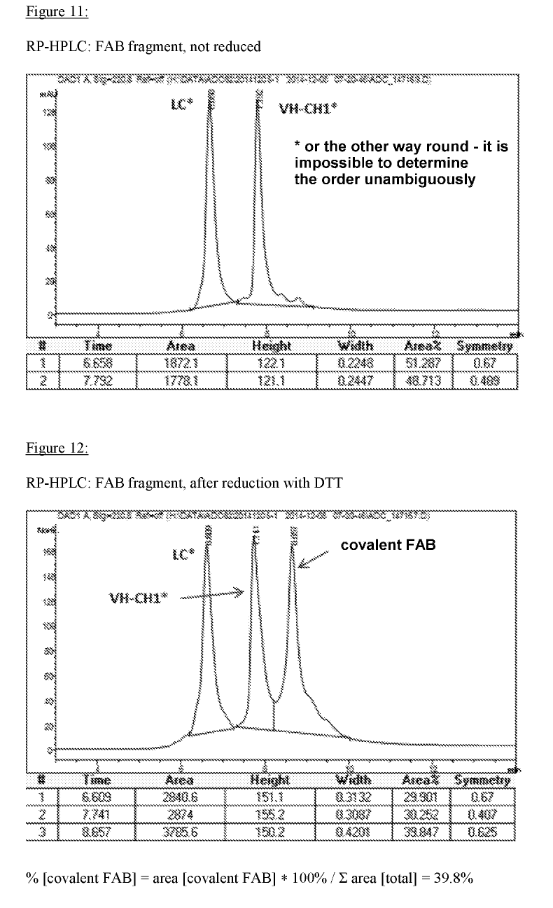

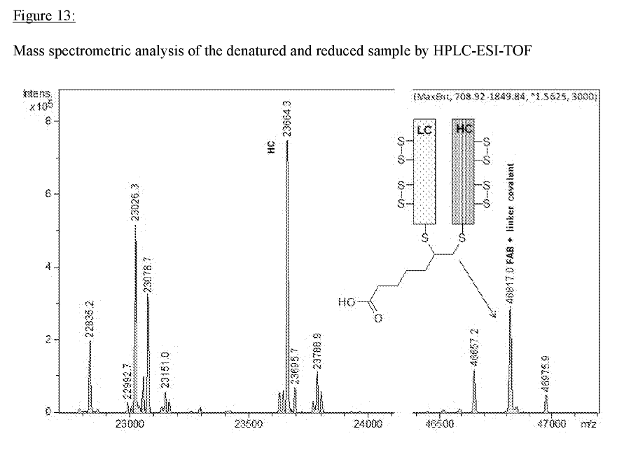

The detected peaks were assigned by retention time comparison with the light chain (LO) and the heavy chain (VH-CH1=H0) of the non-conjugated FAB fragment. The signal detected exclusively in the conjugated sample was assigned to the covalent non-reducibly attached FAB. The percentage of covalently attached FAB was calculated from the signal areas determined by integration. To this end, the quotient of the signal area of the FAB fragment to the total area of all signals was formed and multiplied by 100. The resulting chromatograms and the calculated percentages are shown in FIGS. 11 and 12.

To confirm the covalently attached FAB fragment, the denatured and reduced sample was, after online desalination on a Grom-Sil 300 Butyl-1St column (particle size 5 μm, column dimensions 5 mm×500 μm), analyzed by mass spectrometry using HPLC-ESI-TOF (Impact HD, Bruker Daltonik). The flow rate was 5 μl/min, with the following mobile phase system: mobile phase A: 0.1% formic acid in water, mobile phase B: 0.1% formic acid in 80% isopropanol, 10% acetonitrile and 10% water; gradient: 0 min 22% B, 8 min 22% B, 10 min 24% B, 12 min 80% B, 18 min 95% B, 27 min 95% B, 30 min 22% B.

The spectra obtained for the TIC (total ion chromatogram) signal were added and the molecular weight of the different species was calculated based on MaxEnt deconvolution. By comparison of the masses obtained with the theoretical masses of light chain and heavy chain (VH-CH1) and the covalently attached FAB fragment, it was possible to confirm unambiguously the desired covalent attachment of the FAB fragment. The resulting spectrum is shown in FIG. 13.

### B. LITERATURE REFERENCES

AIMETTI, A. A., FEAVER, K. R. & ANSETH, K. S., 2010. Synthesis of cyclic, multivalent Arg-Gly-Asp using sequential thiol-ene/thiol-yne photoreactions. Chem Commun (Camb), 46, 5781-3.
BALDWIN, A. D. & KIICK, K. L., 2011. Tunable degradation of maleimide-thiol adducts in reducing environments. Bioconjug Chem, 22, 1946-53.
CASTANEDA, L., MARUANI, A., SCHUMACHER, F. F., MIRANDA, E., CHUDASAMA, V., CHESTER, K. A., BAKER, J. R., SMITH, M. E. & CADDICK, S., 2013. Acid-cleavable thiomaleamic acid linker for homogeneous antibody-drug conjugation. Chem Commun (Camb), 49, 8187-9.
CHARI, R. V., MILLER, M. L. & WIDDISON, W. C., 2014. Antibody-drug conjugates: an emerging concept in cancer therapy. Angew Chem Int Ed Engl, 53, 3796-827.
FLYGARE, J. A., PILLOW, T. H. & ARISTOFF, P., 2013. Antibody-drug conjugates for the treatment of cancer. Chem Biol Drug Des, 81, 113-21.
GARNETT, M. C., 2001. Targeted drug conjugates: principles and progress. Adv Drug Deliv Rev, 53, 171-216.
GERONA-NAVARRO, G., YOEL, R., MUJTABA, S., FRASCA, A., PATEL, J., ZENG, L., PLOTNIKOV, A. N., OSMAN, R. & ZHOU, M. M., 2011. Rational design of cyclic peptide modulators of the transcriptional coactivator CBP: a new class of p53 inhibitors. J Am Chem Soc, 133, 2040-3.
GHOSH, S. S., KAO, P. M., MCCUE, A. W. & CHAPPELLE, H. L., 1990. Use of maleimide-thiol coupling chemistry for efficient syntheses of oligonucleotide-enzyme conjugate hybridization probes. Bioconjug Chem, 1, 71-6.
GONG, T., ADZIMA, B. J. & BOWMAN, C. N., 2013. A novel copper containing photoinitiator, copper(II) acylphosphinate, and its application in both the photomediated CuAAC reaction and in atom transfer radical polymerization. Chem Commun (Camb), 49, 7950-2.
JONES, L. H., MCKNIGHT, A. J. & EDITORS, 2013. Biotherapeutics: Recent Developments Using Chemical and Molecular Biology. [In: RSC Drug Discovery Ser., 2013; 36], RSC.
JONES, M. W., STRICKLAND, R. A., SCHUMACHER, F. F., CADDICK, S., BAKER, J. R., GIBSON, M. I. & HADDLETON, D. M., 2012. Polymeric dibromomaleimides as extremely efficient disulfide bridging bioconjugation and pegylation agents. J Am Chem Soc, 134, 1847-52.
KLINGUER-HAMOUR, C., STROP, P., SHAH, D. K., DUCRY, L., XU, A. & BECK, A., 2014. World Antibody-Drug Conjugate Summit, Oct. 15-16, 2013, San Francisco, Calif. MAbs, 6, 18-29.
KRANNIG, K.-S., HUANG, J., HEISE, A. & SCHLAAD, H., 2013. Photochemical thiol-yne functionalization of polypeptide scaffolds. Polymer Chemistry, 4, 3981-3986.
LIBERATORE, F. A., COMEAU, R. D., MCKEARIN, J. M., PEARSON, D. A., BELONGA, B. Q., 3RD, BROCCHINI, S. J., KATH, J., PHILLIPS, T., OSWELL, K. & LAWTON, R. G., 1990. Site-directed chemical modification and cross-linking of a monoclonal antibody using equilibrium transfer alkylating cross-link reagents. Bioconjug Chem, 1, 36-50.
LO CONTE, M., PACIFICO, S., CHAMBERY, A., MARRA, A. & DONDONI, A., 2010. Photoinduced addition of glycosyl thiols to alkynyl peptides: use of free-radical thiol-yne coupling for post-translational double-glycosylation of peptides. J Org Chem, 75, 4644-7.
LO CONTE, M., STADERINI, S., MARRA, A., SANCHEZ-NAVARRO, M., DAVIS, B. G. & DONDONI, A., 2011. Multi-molecule reaction of serum albumin can occur through thiol-yne coupling. Chem Commun (Camb), 47, 11086-8.
MASSI, A. & NANNI, D., 2012. Thiol-yne coupling: revisiting old concepts as a breakthrough for up-to-date applications. Org Biomol Chem, 10, 3791-807.
MILLER, L. W. & CORNISH, V. W., 2005. Selective chemical labeling of proteins in living cells. Current Opinion in Chemical Biology, 9, 56-61.
MINOZZI, M., MONESI, A., NANNI, D., SPAGNOLO, P., MARCHETTI, N. & MASSI, A., 2011. An insight into the radical thiol/yne coupling: the emergence of arylalkyne-tagged sugars for the direct photoinduced glycosylation of cysteine-containing peptides. J Org Chem, 76, 450-9.
RAMIL, C. P. & LIN, Q., 2013. Bioorthogonal chemistry: strategies and recent developments. Chemical Communications, 49, 11007-11022.
ROBERTS, M. J., BENTLEY, M. D. & HARRIS, J. M., 2002. Chemistry for peptide and protein PEGylation. Adv Drug Delivery Rev, 54, 459-476.
SAPRA, P., HOOPER, A. T., O'DONNELL, C. J. & GERBER, H. P., 2011. Investigational antibody drug conjugates for solid tumors. Expert Opin Investig Drugs, 20, 1131-49.
SCHUMACHER, F. F., SANCHANIA, V. A., TOLNER, B., WRIGHT, Z. V., RYAN, C. P., SMITH, M. E., WARD, J. M., CADDICK, S., KAY, C. W., AEPPLI, G., CHESTER, K. A. & BAKER, J. R., 2013. Homogeneous antibody fragment conjugation by disulfide bridging introduces ‘spinostics’. Sci Rep, 3, 1525.
SHEN, B. Q., XU, K., LIU, L., RAAB, H., BHAKTA, S., KENRICK, M., PARSONS-REPONTE, K. L., TIEN, J., YU, S. F., MAI, E., LI, D., TIBBITTS, J., BAUDYS, J., SAAD, O. M., SCALES, S. J., MCDONALD, P. J., HASS, P. E., EIGENBROT, C., NGUYEN, T., SOLIS, W. A., FUJI, R. N., FLAGELLA, K. M., PATEL, D., SPENCER, S. D., KHAWLI, L. A., EBENS, A., WONG, W. L., VANDLEN, R., KAUR, S., SLIWKOWSKI, M. X., SCHELLER, R. H., POLAKIS, P. & JUNUTULA, J. R., 2012. Conjugation site modulates the in vivo stability and therapeutic activity of antibody-drug conjugates. Nat Biotechnol, 30, 184-9.
TIAN, F., LU, Y., MANIBUSAN, A., SELLERS, A., TRAN, H., SUN, Y., PHUONG, T., BARNETT, R., HEHLI, B., SONG, F., DEGUZMAN, M. J., ENSARI, S., PINKSTAFF, J. K., SULLIVAN, L. M., BIROC, S. L., CHO, H., SCHULTZ, P. G., DIJOSEPH, J., DOUGHER, M., MA, D., DUSHIN, R., LEAL, M., TCHISTIAKOVA, L., FEYFANT, E., GERBER, H. P. & SAPRA, P., 2014. A general approach to site-specific antibody drug conjugates. Proc Natl Acad Sci USA, 111, 1766-71.
TODA, N., ASANO, S. & BARBAS, C. F., 3^{RD}, 2013. Rapid, stable, chemoselective labeling of thiols with Julia-Kocienski-like reagents: A serum-stable alternative to maleimide-based protein conjugation. Angew Chem Int Ed Engl, 52, 12592-6.
VERONESE, F. M. & MERO, A., 2008. The impact of PEGylation on biological therapies. BioDrugs, 22, 315-29.
WIDDISON, W. C., WILHELM, S. D., CAVANAGH, E. E., WHITEMAN, K. R., LEECE, B. A., KOVTUN, Y., GOLDMACHER, V. S., XIE, H., STEEVES, R. M., LUTZ, R. J., ZHAO, R., WANG, L., BLATTLER, W. A. & CHARI, R. V., 2006. Semisynthetic maytansine analogues for the targeted treatment of cancer. J Med Chem, 49, 4392-408.
WU, A. M. & SENTER, P. D., 2005. Arming antibodies: prospects and challenges for immunoconjugates. Nat Biotechnol, 23, 1137-46.
ZHOU, W., ZHENG, H., LI, Y. & LIU, H., 2010. Synthesis of sulfuric macrocycles and a rotaxane through thiol-yne click and dithiol coupling reactions. Org Lett, 12, 4078-81.

1. Process for preparing peptide or protein conjugates pairwise C2-bridged via cysteine amino acids, wherein a peptide or protein of the formula (II) 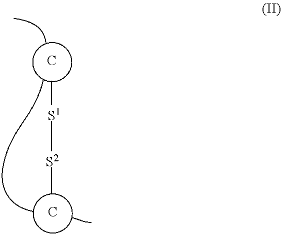
in which S¹ and S² represent cysteine sulphur atoms of this peptide or protein which are bonded in a disulphide bridge is converted under reducing conditions into a peptide or protein of the formula (III) 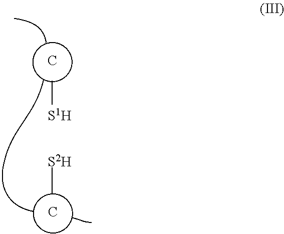
and this is then reacted under free-radical reaction conditions with an alkyne derivative of the formula (IV) 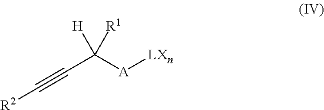
in which
R¹ and R² independently of one another represent hydrogen, alkyl, cycloalkyl, heterocycloalkyl, aryl, heteroaryl, hydroxy, alkoxy, amino, alkylamino, dialkylamino, hydroxycarbonyl, alkoxycarbonyl, alkylcarbonylamino or alkoxycarbonylamino,
where alkyl, cycloalkyl, heterocycloalkyl, aryl, heteroaryl, alkoxy, alkylamino, dialkylamino, alkoxycarbonyl, alkylcarbonylamino and alkoxycarbonylamino for their part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino,
A represents a bond or a hydrocarbon chain having 1 to 100 carbon atoms from alkylene, cycloalkylene and/or arylene groups which may be interrupted once or more than once by identical or different groups selected from the group consisting of —O—, —S—, —S(═O)—, —S(═O)₂—, —NH—, —N(CH₃)—, —C(═O)—, —NH—C(═O)—, —C(═O)—NH—, —O—C(═O)—, —C(═O)—O—, —SO₂—NH—, —NH—SO₂—, —NH—NH—, —SO₂—NH—NH—, —NH—NH—SO₂—, —C(═O)—NH—NH—, —NH—NH—C(═O)—, —NH—C(═O)—NH—, —O—C(═O)—NH—, —NH—C(═O)—O— and a 4- to 10-membered aromatic or non-aromatic heterocycle having up to 4 heteroatoms from the group consisting of N, O, S, S(═O) and S(═O)₂,
or
R² and A are attached to one another and together with the interjacent carbon atoms form an 8-membered carbocycle which may be fused to a 3- to 6-membered cycloalkyl ring,
where the 8-membered carbocycle and optionally the fused cycloalkyl ring may be mono- or polysubstituted by identical or different substituents from the group consisting of fluorine, alkyl, hydroxy, hydroxyalkyl and alkoxy,
L represents a bond or a linker,
X may be present n-times and represents an active compound molecule, a polymer, an alkaloid, a peptide, a protein, a carbohydrate, a nucleotide, a nucleoside, a steroid, a terpene, a porphyrin, a chlorin, a corrin, an eicosanoid, a pheromone, a vitamin, a biotin, a dye molecule or a cryptand or represents hydrogen, hydroxy, alkoxy, amino, alkylamino, dialkylamino, hydroxycarbonyl, alkoxycarbonyl, alkylcarbonylamino, alkoxycarbonylamino, alkyl, cycloalkyl, heterocycloalkyl, aryl or heteroaryl,
where alkyl for its part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino
and
where cycloalkyl, heterocycloalkyl, aryl and heteroaryl for their part may be mono- or polysubstituted by identical or different substituents from the group consisting of halogen, alkyl, hydroxy, alkoxy, amino, alkylamino, dialkylamino, hydroxycarbonyl, alkoxycarbonyl, alkylcarbonylamino and alkoxycarbonylamino,
and
n represents an integer in the range from 1 to 10 inclusive,
wherein in the case that more than one group X is present their individual meanings can be identical or different,
to give a conjugate of the formula (I) 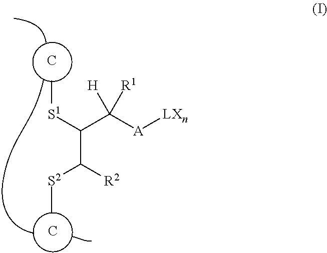
in which R¹, R², A, L, X and n have the meanings given above.

2. Peptide or protein conjugate of the general formula (I) 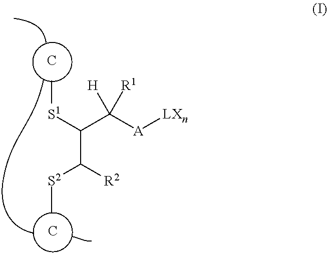
in which
S¹ and S² represent cysteine sulphur atoms, previously bonded in a disulphide bridge, of a peptide or protein,
R¹ and R² independently of one another represent hydrogen, alkyl, cycloalkyl, heterocycloalkyl, aryl, heteroaryl, hydroxy, alkoxy, amino, alkylamino, dialkylamino, hydroxycarbonyl, alkoxycarbonyl, alkylcarbonylamino or alkoxycarbonylamino,
where alkyl, cycloalkyl, heterocycloalkyl, aryl, heteroaryl, alkoxy, alkylamino, dialkylamino, alkoxycarbonyl, alkylcarbonylamino and alkoxycarbonylamino for their part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino,
A represents a bond or a hydrocarbon chain having 1 to 100 carbon atoms from alkylene, cycloalkylene and/or arylene groups which may be interrupted once or more than once by identical or different groups selected from the group consisting of —O—, —S—, —S(═O)—, —S(═O)₂—, —NH—, —N(CH₃)—, —C(═O)—, —NH—C(═O)—, —C(═O)—NH—, —O—C(═O)—, —C(═O)—O—, —SO₂—NH—, —NH—SO₂—, —NH—NH—, —SO₂—NH—NH—, —NH—NH—SO₂—, —C(═O)—NH—NH—, —NH—NH—C(═O)—, —NH—C(═O)—NH—, —O—C(═O)—NH—, —NH—C(═O)—O— and a 4- to 10-membered aromatic or non-aromatic heterocycle having up to 4 heteroatoms from the group consisting of N, O, S, S(═O) and S(═O)₂,
or
R² and A are attached to one another and together with the interjacent carbon atoms form an 8-membered carbocycle which may be fused to a 3- to 6-membered cycloalkyl ring,
where the 8-membered carbocycle and optionally the fused cycloalkyl ring may be mono- or polysubstituted by identical or different substituents from the group consisting of fluorine, alkyl, hydroxy, hydroxyalkyl and alkoxy,
L represents a bond or a linker,
X may be present n-times and represents an active compound molecule, a polymer, an alkaloid, a peptide, a protein, a carbohydrate, a nucleotide, a nucleoside, a steroid, a terpene, a porphyrin, a chlorin, a corrin, an eicosanoid, a pheromone, a vitamin, a biotin, a dye molecule or a cryptand or represents hydrogen, hydroxy, alkoxy, amino, alkylamino, dialkylamino, hydroxycarbonyl, alkoxycarbonyl, alkylcarbonylamino, alkoxycarbonylamino, alkyl, cycloalkyl, heterocycloalkyl, aryl or heteroaryl,
where alkyl for its part may be mono- or polysubstituted by identical or different substituents from the group consisting of halogen, hydroxy, alkoxy, amino, alkylamino, dialkylamino, hydroxycarbonyl, alkoxycarbonyl, alkylcarbonylamino and alkoxycarbonylamino
and
where cycloalkyl, heterocycloalkyl, aryl and heteroaryl for their part may be mono- or polysubstituted by identical or different substituents from the group consisting of halogen, alkyl, hydroxy, alkoxy, amino, alkylamino, dialkylamino, hydroxycarbonyl, alkoxycarbonyl, alkylcarbonylamino and alkoxycarbonylamino,
and
n represents an integer in the range from 1 to 10 inclusive,
wherein in the case that more than one group X is present their individual meanings can be identical or different.

3. Peptide or protein conjugate as defined in claim 2 for the diagnosis and/or treatment of disorders.

4. Peptide or protein conjugate as defined in claim 2 for use in a method for the diagnosis and/or treatment of cancer and tumour disorders.

5. Pharmaceutical composition comprising a peptide or protein conjugate as defined in claim 2 in combination with one or more inert non-toxic pharmaceutically suitable auxiliaries.

6. Pharmaceutical composition according to claim 5 for the diagnosis and/or treatment of cancer and tumour disorders.

7. Method for the diagnosis and/or treatment of cancer and tumour disorders utilizing a peptide or protein conjugate as defined in claim 2.
